(19) United States
(12) Patent Application Publication
(10) Pub. No.: US 2012/0208712 A1
(43) Pub. Date: Aug. 16, 2012

(54) SIRTUIN 5 POLYMORPHISMS AND NEUROLOGICAL DISEASES

(75) Inventors: Etienne Sibille, Harmony, PA (US); Christin Glorioso, Pittsburgh, PA (US)

(73) Assignee: The University of Pittsburgh - Of the Commonwealth System of Higher Education

(21) Appl. No.: 13/194,534

(22) Filed: Jul. 29, 2011

Related U.S. Application Data
(60) Provisional application No. 61/368,879, filed on Jul. 29, 2010.

(51) Int. Cl. C40B 30/04; C07H 21/04; C12Q 1/68

(52) U.S. Cl. 506/9; 435/6.11; 536/24.31

(57) ABSTRACT

A method for determining a subject's risk of developing a neurological disease or disorder such as Huntington's or Parkinson's disease, based on the presence of SIRT5ₚᵣₒₘ₂ (rs9382222) C/C genotype is provided. Also provided are compositions including primers and probes, which are capable of selectively interacting with nucleic acids, such as those comprising the SNP disclosed herein.

### CROSS-REFERENCE TO RELATED APPLICATIONS

This application claims benefit of and priority to U.S. Provisional Application No. 61/368,879 filed on Jul. 29, 2010, which is incorporated by reference in its entirety.

### STATEMENT REGARDING FEDERALLY SPONSORED RESEARCH OR DEVELOPMENT

This invention was made with government support under the following grants: KO1 MH067721, KO2 MH084060, 1 R01 MH077159-01, and F30 AG030325, awarded by the National Institutes of Health. The government has certain rights in the invention.

### REFERENCE TO SEQUENCE LISTING

The Sequence Listing submitted Jul. 29, 2011 as a text file named “UNIPIT_{—}02087_ST25.txt”, created on Jul. 29, 2011, and having a size of 4,096 bytes is hereby incorporated by reference.

### FIELD OF THE INVENTION

The invention relates to methods and compositions for assessing a subject's risk or propensity of developing a disease, particularly neurological diseases such as Parkinson's disease and Huntington's disease.

### BACKGROUND OF THE INVENTION

Disease-specific ages of onset are core features of many neurological disorders, ranging from late-onset neurodegenerative diseases such as Alzheimer's and Parkinson's diseases (average onset 60 and 75 years, respectively) (Nussbaum, et al., N Engl J Med., 348:1356-64 (2003)) to earlier onset psychiatric disorders such as schizophrenia and bipolar disorder (average onset 25 years) (Tsuang, et al., Textbook in Psychiatric Epidemiology, Wiley, Weinheim, Germany, 2002). The mechanism(s) underlying age thresholds and the factors that contribute to individual variability in ages of onset within diseases are largely unknown. Studies have predominantly focused on contrasting diseased brains with age-matched controls, a strategy that may be problematic, as it is becoming increasingly evident that normal aging is an integral aspect and modulator of disease onset and progression. Evidence for this comes from the sheer prevalence of diseases with increasing age, such as Alzheimer's disease, which increases exponentially from age 75 upward reaching nearly 45% by age 95 (Nussbaum, et al., N Engl J Med., 348:1356-64 (2003)).

Robust morphological and molecular changes progressively occur in the normal aging brain throughout adulthood and into old age (Yankner, et al., Annu Rev Pathol., 3:41-66 (2008)). Morphological changes include progressive loss of grey matter density (Resnick, et al., J Neurosci., 23:3295-301 (2003)), disrupted myelination, and increasing reactive gliosis. These changes reflect dendritic shrinkage, synaptic loss, (Morrison, et al., Science, 278:412-9, (1997); Yankner, et al., Annu Rev Pathol., 3:41-66 (2008)) and thickening glial processes (glial dystrophy) (Conde, et al., J Neuropathol Exp Neurol. 65:199-203 (2006)). Within neurons, increased DNA damage and reactive oxygen species, calcium dysregulation, mitochondrial dysfunction and inflammatory processes have been reported (Reviewed in Yankner, et al., Annu Rev Pathol., 3:41-66 (2008)). Several groups have characterized the molecular underpinnings of these changes using human post-mortem brain microarray (Berchtold, et al., Proc Natl Acad Sci USA., 105:15605-10 (2008); Erraji-Benchekroun, et al., Biol Psychiatry, 57:549-58 (2005); Lu, et al., Nature, 429:883-91 (2004)); however, no systematic effort has been undertaken to explore the molecular overlap of normal aging and disease pathways. Although studies have shown that molecular age accurately predicts chronological age (Erraji-Benchekroun, et al., Biol Psychiatry, 57:549-58 (2005)), there are no studies relating to the genetic control of observed deviations from predicted molecular age trajectory, or gene variants which may affect rates of molecular brain aging and risk for neurological diseases, through overlapping age-related and disease pathways.

Recent studies have revealed an increasing role of the sirtuin gene family in neurodegenerative disease (Gan, et al., Neuron, 58:10-4 (2008)). The sirtuin family of protein deacetylating enzymes belongs to a set of proteins that act as metabolic sensors and contribute to the complex process of organism aging. Mammals have seven isoforms of Sirtuin, three of them, SIRT3, SIRT4 and SIRT5, are localized to the mitochondria. WO 2008/060400 discloses SIRT1 polymorphic variants, that could contribute to or be predictive of the development of diseases or discorders related to aging, diabetes, obesity, neurodegenerative diseases, etc. Studies have shown altered SIRT5 expression in htr1b^{KO} mouse cortex, a mouse model with anticipated brain aging (Sibille, et al., Mol Psychiatry, 12:1042-56, 975 (2007)). Sirt5 is a single copy gene situated on chromosome locus 6p23. Analyses of expressed sequence tag databases indicate that Sirt5 is predominantly expressed in lymphoblasts, heart muscle cells, thymus, brain, liver, kidney, and skeletal muscle (Reviewed in Gertz, et al., Biochimicha et Biophysica Acta, 1804:1658-1665 (2010).

It is an object of the present invention to provide methods and compositions for determining a subject's risk of developing a neurological disorder.

It is also an object of the present invention to provide probes and primers for detecting the presence of SIRT5ₚᵣₒₘ₂ (rs9382222) genotypes in a biological sample.

It is further an object of the present invention to provide methods for detecting the presence of SIRT5ₚᵣₒₘ₂ (rs9382222) SNP in a biological sample.

### SUMMARY OF THE INVENTION

Methods and compositions for determining a subject's risk of developing a neurological disease or disorder, a mitochondrial dysfunction-related disorder, or a combination thereof are provided. Exemplary methods included determining the genotype of SIRT5ₚᵣₒₘ₂ (rs9382222). It has been discovered that the presence of SIRT5ₚᵣₒₘ₂ (rs9382222) C/C genotype is indicative of an increased risk of developing a neurological disease or disorder, a mitochondrial dysfunction-related disorder, or a combination thereof, relative to the presence of the SIRT5ₚᵣₒₘ₂ (rs9382222) C/T genotype. Subjects having the C/C genotype at the SIRT5 SNP (rs9382222) display poorer function on cognitive function tests (lower DSST score) and motor function tests (longer time to walk 20 meters), and have increased self-reported symptoms of a depressed mood (higher CES-D score), as compared to all other subjects, for example subjects having the C/T genotype for SIRT5ₚᵣₒₘ₂ (rs9382222).

Representative neurological disorders linked to mitochondrial dysfunction include, but are not limited to Huntington's disease, Parkinson's disease, Alzheimer's, amyotrophic lateral sclerosis, schizophrenia and bipolar disorder. The method includes detecting the presence of SIRT5ₚᵣₒₘ₂ SNP (rs9382222) C/C or C/T genotypes in a biological sample obtained from the subject. Another embodiment provides detecting the presence or absence of SIRT5ₚᵣₒₘ₂ SNP (rs9382222) T allele or T/T genotype.

Also provided are compositions including nucleic acid primers and probes capable of specifically detecting SIRT5ₚᵣₒₘ₂ SNP (rs9382222) C/C or C/T genotypes. In a preferred embodiment, the probe hybridizes under stringent conditions to a region of SEQ ID NO:1 including the C nucleotide at position 27, wherein SEQ ID NO:1 comprises CCACTAAACTCCCTCCTACCCCCACCCAATAACTATGGACAACTTTCC ATCC (SEQ ID NO:1) and wherein the probe does not hybridize under stringent conditions to an oligonucleotide having a nucleic acid sequence of CCACTAAACTCCCTCCTACCCCCACCTAATAACTATGGACAACTTTCC ATCC (SEQ ID NO:2).

Also provided herein are kits for detecting the presence of SIRT5ₚᵣₒₘ₂ (rs9382222) SNP, preferably the C or T allele, in a biological sample. Detection kits and systems, include but are not limited to, packaged probe and primer sets, arrays/microarrays of nucleic acid molecules, and beads that contain one or more probes, primers, or other detection reagents for detecting the disclosed SNP.

### BRIEF DESCRIPTION OF THE DRAWINGS

FIGS. 1A-D are plots of molecular ages by depression status of different brain areas. Anterior cingulate cortex (ACC); amygdila (AMY); prefrontal cortex (PFC); Brodmann area 9 (BA9); Brodmann area 47 (BA47) for FIGS. 1A, 1B, 1C, and 1D, respectively. “C” and “D” refer to Control and Depressed group averages for (molecular age-chronological age) and p-values were generated from performing two group t-tests on these values. Molecular ages were calculated using the cross-area age biosignature.

FIGS. 2A-F are representative age-regression gene plots conserved molecular aging profiles across human brain areas. FIGS. 2G-V are cross area comparisons of age-related gene expression changes [70-20 yrs change; ordered from most increased with age (plots on positive side of zero) to most decreased with age (plots on negative side of zero)]; n=number of age-regulated genes; R=directed Pearson coefficient. Up-regulated reactive gliosis markers (GFAP), down-regulated growth factors (BDNF and IGF-1), synaptic markers (SYN2); calcium homeostasis genes (CALB-1); neuronal-specific transcripts (NRSN2); Anterior cingulate cortex (ACC); amygdila (AMY); prefrontal cortex (PFC); Brodmann area 9 (BA9); Brodmann area 47 (BA47).

FIG. 3 is a plot of the correlation of quantitative PCR and microarray quantification of gene expression levels. QPCR validation of a set of 42 age-regulated (p<0.01) genes in total confirm results of Erraji-Benchekroun, L., et al., Biol Psychiatry, 57:549-558 (2005). Results were re-analyzed for age effects and converted to percent change over 50 years of (Age 70-Age 20) for comparability of results to current array data results.

FIG. 4A-B are plots showing the molecular ages of subjects calculated using ACC (n=4443) or AMY (n=2820) specific age-regulated genes (p<0.01).

FIG. 5 is a diagram illustrating the chromosomal context of human Sirt5 including up to 5 kb upstream of the transcriptional start site. Areas of homology between mouse and human DNA (boxes on chromosome) and putative promoter regions (stars) are also shown. The Sirt5 prom 1, 2, and 3 snps (leftmost to rightmost) are shown in the rectangular box.

FIG. 6 is a plot showing molecular age versus chronological age of human subjects.

FIG. 7A is a pie graph showing the percentage of age and non age-regulated genes identified as neurological disease-related. Left: n=1,098 for neurological disease-related age-regulated genes. Alzheimer's Disease (AD) (n=185), Parkinson's Disease (PD) (n=170), Huntington's Disease (HD) (n=267), Amyotrophic Lateral Schlerosis (ALS) (n=164), Schizophrenia (SCZ) (n=161), and Bipolar Disorder (BPD) associated genes (n=285). Right: n=321 for non-age regulated genes that are neurological disease-related with no specific diseases identified. FIG. 7B shows example plots of age-regulated disease-related genes. Transcription levels of the indicated gene (percent at age 20) are plotted against age in years. Trendlines are best-fit regression lines for ACC, Amygdala, PFC BA9, and PFC BA47 with equations and corresponding regression p-values.

FIG. 8 is a bar graph showing Top 20 Ingenuity® Functional Categories associated with age-regulated genes (figure adapted from Ingenuity®). Criteria for selection for age regulated genes were age-regression p<0.001 in at least one area or p<0.01 in two brain areas (n=3,935).

FIGS. 9A-9F shows the direction (up or down) of change in expression of age-regulated genes associated with the top six neurological diseases. FIG. 9A shows age-related genes associated with Bipolar Disorder (285 Genes); FIG. 9B shows age-related genes associated with Huntington's Disease (267 Genes); FIG. 9C shows age-related genes associated with Alzheimer's Disease (185 Genes); FIG. 9D shows age-related genes associated with Parkinson's Disease (170 Genes); FIG. 9E shows age-related genes associated with Amyotrophic Lateral Sclerosis (164 Genes); and FIG. 9F shows age-related genes associated with Schizophrenia (161 Genes).

FIG. 10 is a bar graph showing top 20 Ingenuity® functional categories analysis of genes that were not age-regulated. Criteria for non-age-regulated genes were p>0.05 in all four brain areas (n=7790).

FIGS. 11A-11E show SIRT5ₚᵣₒₘ₂ effects on anterior cigulate cortex (ACC) molecular aging. FIG. 11A is a bar graph showing SIRT5 expression in ACC by prom2 genotype. Arbitrary transcriptional levels are shown pairwise for C/T (left bar) or C/C (right bar) expression. FIG. 11B is a Venn diagram (ACC) of age (p<0.01) and SIRT5ₚᵣₒₘ₂ (p<0.01) associated transcripts; the (number yr)=average number of molecular years greater in C/C subjects than C/T (left); and plots of molecular ages of subjects by SIRT5ₚᵣₒₘ₂ genotype based on all age-regulated transcripts (top-right) (C/C-slope=1.13; C/T-slope=0.94) and core transcripts (bottom-right) (C/C-slope=1.26; C/T-slope=0.77). FIG. 11C is a schematic of mitochondrial age-regulated genes with accelerated age-trajectories in SIRT5-low-expresser subjects with age down-regulated transcripts and one group of age-upregulated transcripts; HD=Huntington's disease-associated genes; PD=Parkinson's diseases-associated genes. FIG. 11D are graphs showings the representative core transcript age-regressions of Pink-1 (left) or DJ1 (right) by SIRT5ₚᵣₒₘ₂ genotype: Arbitrary transcript levels are plotted against age in years. FIG. 11E graph showing a multi-hit model of age of onset. Percent disease gene transcript levels is plotted against age in years.

FIG. 12 is a bar graph showing quantitative PCR of Sirt5 expression (arbitrary units) by Sirtₚᵣₒₘ₂ genotype in AMY.

FIG. 13 is a bar graph showing Ingenuity® Functional Categories significantly affected by Sirt5ₚᵣₒₘ₂ genotype.

FIG. 14 is a bar graph showing Ingenuity® Canonical Pathways significantly affected by Sirt5ₚᵣₒₘ₂ intersection transcripts in ACC (n=231).

FIG. 15 shows the direction (up or down) of change in expression of Huntington's and Parkinson's associated genes affected by Sirt5ₚᵣₒₘ₂ genotype in ACC.

FIG. 16 shows QPCR validation of Pink1 and DJ-1 expression differences by Sirt5ₚᵣₒₘ₂ genotype. Arbitrary transcriptional levels are shown pairwise for C/T (left bar) or C/C (right bar) expression. * denotes genotypic expression difference p<0.05 and ** denotes genotypic expression difference p<0.01.

### DETAILED DESCRIPTION OF THE INVENTION

### Definitions

As used herein, the term “single nucleotide polymorphism” (SNP) refers to a variation of a single nucleotide.

As used herein, the terms “probe” or “primer” refer to a nucleic acid or oligonucleotide that forms a hybrid structure with a sequence in a target region of a nucleic acid due to complementarity of the probe or primer sequence to at least one portion of the target region sequence.

As used herein, SIRT5ₚᵣₒₘ₂ SNP detection “kits” and “systems”, refers to combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, etc.).

The term “stringent hybridization conditions” as used herein means that hybridization will generally occur if there is at least 95% and preferably at least 97% sequence identity between the probe and the target sequence. Examples of stringent hybridization conditions are overnight incubation in a solution including 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C. Other hybridization and wash conditions are well known and are exemplified in Sambrook, et al, Molecular Cloning: A Laboratory Manual, Third Edition, Cold Spring Harbor, N.Y. (2000), particularly chapter 11.

As used herein, the term “neurological disease or disorder” refers to a disease or disorder of the nervous system including but is not limited to Huntington's disease, Parkinson's disease, Alzheimer's disease, amyotrophic lateral sclerosis, schizophrenia and bipolar disorder.

As used herein the term “mitochondrial dysfunction” refers to reduced or impaired mitochondrial function relative to healthy subjects resulting in one or more symptoms of a disease or disorder.

As used herein the term “biological sample” refers to a sample obtained from a subject, wherein the sample contains genomic DNA.

### I. Method for Assessing Risk of Developing a Neurological or Mitochondrial Related Disease

Using microarray analysis of four human brain areas in two cohorts, it has been discovered that neurological disease pathways largely overlap with molecular aging, and that subjects carrying a newly-characterized low-expressing polymorphism in a candidate longevity gene (Sirtuin5; SIRT5ₚᵣₒₘ₂) have older brain molecular ages, potentially through accelerated decline of mitochondrial function with age. SIRT5ₚᵣₒₘ₂ (rs9382222) was identified as a SNP of interest due to its location in a mouse/human conserved region predicted by two separate programs to contain a promoter region. The SIRT5ₚᵣₒₘ₂ SNP can be used as a biomarker to identify subjects at risk of developing neurological or mitochondrial dysfunction-related diseases. In a preferred embodiment, the SIRT5ₚᵣₒₘ₂ genotype is used to identify a subject at risk of developing a neurological disorder such as Huntington's disease, Parkinson's disease, Alzheimer's disease, amyotrophic lateral sclerosis, schizophrenia and bipolar disorder.

A. Surtuin 5 (SIRT 5)

SIRT5 is an endogenous protein localized in the matrix of the mitochondria. It is located specifically in the mitochondria intermembrane space. It has a 36 amino acid residue, N-terminal mitochondrial targeting signal which is removed once in the mitochondria. SIRT5 is expressed in multiple tissues: brain, muscle, heart, liver and kidney.

SIRT5 has a deacetylase function. SIRT5 acts on acetylated histones or BSA28 against acetylated histone H4 peptide, showing its deacetylase activity, and against acetylated cytochrome C30, intermembrane mitochondrial space protein.

Biologically, carbamoil phosphate synthase (CPS1)—a mitochondrial matrix enzyme—has been identified as a substrate for SIRT5. CPS1 plays an important role in urea synthesis in the urea cycle. In fact, this enzyme acts as a rate-limiting enzyme modulating urea synthesis. Specifically, CPS1 removes the ammonia generated by amino acid catabolism. SIRT5 increases CPS1 activity by stimulating the deacetylation function of CPS1 with NAD+. Thus, SIRT5 increases the urea formation in conditions when the nutrient intakes are low, the ammonia generation is high, and the amino acid catabolism is also high. A loss of ammonia is seen in metabolism with low calorie intake and high-protein diet (HPD), and this is when SIRT 5 regulates CPS1 [11].

A reduced calorie condition is a circumstance that regulates SIRT5 expression. Once the calorie restriction begins SIRT5 starts to deacetylate CPS1 triggering the activity of CPS1 enzyme; this activation causes the exchange of ammonia in carbamoyl phosphate. This exchange consequently causes the excretion of carbamoyl phosphate as urea in the urea cycle.

New findings have shown a controversy about whether or not SIRT5 increase acetylation and/or hyperacetylation of CPS1 during diets with calorie restrictions. Researchers based this theory on an experiment where, under a low calorie intake diet, they study the acetylation of the CPS1; the results show that 24 sites were acetylated but seven sites were hyperacetylated. In that study no site was found as deacetylated.

Other studies have shown nine acetylating sites in CPS1, but in contrast with other experiments it shows that 4 sites were acetylated during feeding and fasting, another 4 sites were acetylated upon fasting, and one site was deacetylated.

B. Single Nucleotide Polymorphisms (SNP)

The disclosed methods generally include detecting a single nucleotide polymorphism at nucleotide position 27 on SEQ ID NO: 1, and ultimately the genotype of SIRT5ₚᵣₒₘ₂ (rs9382222). A homozygous cytosine genotype rather than a heterozygous genotype at this position is an indication of increased risk of developing a neurological or mitochondrial disease or disorder, for example, Huntington's disease, Parkinson's disease, Alzheimer's disease, amyotrophic lateral sclerosis, schizophrenia and bipolar disorder disease relative to the heterozygous condition, i.e., C/T.

A Single Nucleotide Polymorphism (SNP) is a DNA sequence variation occurring when a single nucleotide—A (adenosine), T (thymine), C (cytosine), or G (guanine)—in the genome (or other shared sequence) differs between members of a species (or between paired chromosomes in an individual). SNPs may fall within coding sequences of genes, noncoding regions of genes, or in the intergenic regions between genes. SNPs within a coding sequence will not necessarily change the amino acid sequence of the protein that is produced, due to degeneracy of the genetic code. A SNP in which both forms lead to the same polypeptide sequence is termed synonymous (sometimes called a silent mutation)—if a different polypeptide sequence is produced they are non-synonymous. SNPs that are not in protein coding regions may still have consequences for gene splicing, transcription factor binding, or the sequence of non-coding RNA.

A subject identified as having an increased risk of developing a neurological or mitochondrial-related disease or disorder includes a subject carrying SIRT5ₚᵣₒₘ₂ (rs9382222) C/C genotype. The method includes the steps analyzing a biological sample obtained from a subject to determine the genotype of SIRT5ₚᵣₒₘ₂ (rs9382222) in the biological sample. Any biological sample that contains genomic DNA of a subject can be employed, including tissue samples and blood samples. The DNA may be isolated from the biological sample prior to genotyping the DNA for SIRT5ₚᵣₒₘ₂ (rs9382222). Methods for genotyping the disclosed SIRT5ₚᵣₒₘ₂ (rs9382222) are provided below. In one embodiment, the genomic DNA of the biological sample is tested for the presence or absence of the SIRT5ₚᵣₒₘ₂ C/C genotype.

C. SNP Detection Methods

A wide variety of techniques have been developed for SNP detection and analysis, see, e.g. U.S. Pat. No. 5,858,659 to Sapolsky, et al. In addition, ligase based methods are described by Barany et al. (1997) WO97/31256 and Chen, et al., Genome Res. 8(5):549-56 (1998); mass-spectroscopy-based methods by Monforte (1998) WO98/12355, Turano, et al. (1998) WO98/14616 and Ross, et al., Anal Chem. 15:4197-202 (1997); PCR-based methods by Hauser, et al., Plant J. 16:117-25 (1998); exonuclease-based methods by Mundy, U.S. Pat. No. 4,656,127; dideoxynucleotide-based methods by Cohen, et al. WO91/02087; Genetic Bit Analysis or GBA™. by Goelet, et al. WO92/15712; Oligonucleotide Ligation Assays or OLAs by Landegren, et al., Science 241:1077-1080 (1988) and Nickerson, et al., Proc. Natl. Acad. Sci. (U.S.A.) 87:8923-8927 (1990); and primer-guided nucleotide incorporation procedures by Prezant, et al., Hum. Mutat. 1:159-164 (1992); Ugozzoli, et al., GATA 9:107-112 (1992); Nyreen, et al., Anal. Biochem. 208:171-175 (1993), which are all hereby incorporated herein by reference for the teaching of SNP detection methods.

The disclosed methods contemplate the use of any method of detecting the SNPs known in the art. For example, the method can include the use of restriction fragment length polymorphism; allele specific hybridization; molecular beacon; allele specific oligonucleotide ligation; rolling circle DNA amplification; mass spectroscopy; gene sequencing, or variations thereof.

1. Allele Specific Hybridization

The provided method can include detecting the SNP by Allele Specific Hybridization. This method relies on selective hybridization to distinguish between two DNA molecules differing by one base. In general, the method involves applying labeled PCR fragments to immobilized oligonucleotides representing SNP sequences. After stringent hybridization and washing conditions, label intensity is measured for each SNP oligonucleotide. Thus, the provided method can include providing a nucleic acid probe that hybridizes under stringent conditions to an oligonucleotide SEQ ID NO:1 but does not hybridize under stringent conditions to an oligonucleotide SEQ ID NO:2, and detecting hybridization of said probe to the nucleic acid sample. The nucleic acid probe can comprise at least 5, 6, 7, 8, 9, 10, 11, 12, 13, 14, 15, 16, 17, 18, 19, 20, 21, 22, 23, 24, 25, 26, 27, 28, 29, or 30 nucleotides that hybridize to SEQ ID NO:1 selectively over SEQ ID NO:2 under stringent conditions.

The probe can include a label such as a fluorescent dye (also known herein as fluorochromes and fluorophores). Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy 492/515; Bodipy 493/503; Bodipy 500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.18; Cy3.5™; Cy3™; Cy5.18; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydrorhodamine 123 (DHR); Dil (DTIC 18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™ (high pH); Fura Red™/Fluo-3; Fura-2; Fura-‘2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type’ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; MonObromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-1 PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl)quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO—PRO3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

2. Single-Step Homogeneous Methods

TaqMan® Gene Expression Assays, molecular beacons, and Scorpion® probe assays are all microtiter plate-based fluorescent readout systems, initially designed for real time PCR expression analyses. TaqMan® assays and molecular beacons both rely on allele-specific hybridization of oligonucleotides during PCR for allele discrimination, while scorpion assays can use either allele-specific PCR or allele-specific hybridization chemistry for allelic discrimination. They all can be performed as an endpoint assay in a completely homogeneous reaction. All the reagents and genomic DNA are mixed at the beginning, and the fluorescent signal is read after the thermocycling step. There is no separate pre-amplification step, or intermediate processing, making them the simplest assay formats possible.

Allelic discrimination using TaqMan® gene expression assays is based on the design of two TaqMan® probes, specific for the wildtype allele and the mutant allele. TaqMan® SNP analysis utilizes the 5′ exonuclease activity of DNA Taq polymerase and the quenching effects of specific florescent dyes to determine the relative frequency of each allele within an individual genome. Primers are designed against a conserved region of the genome flanking the locus of interest. Two probes are designed across the locus of interest, one for each allele. Each probe is labeled with a different reporter dye as well as a quencher molecule. Proximity to the quencher dye inhibits the florescence of the reporter molecule. During thermocycling, the probe anneals to the locus of interest in an allele specific manner. As the Taq DNA polymerase extends the primers, it also degrades the annealed probe, allowing the florescent dye to come out of the sphere of influence of the quencher and thus become detectable.

The provided method can detect the SNP using molecular beacons. Molecular beacons are oligonucleotide probes that can report the presence of specific nucleic acids in homogenous solutions (Tyagi, et al., Nature Biotechnology, 14:303-308 (1996)). Molecular beacons are hairpin shaped molecules with an internally quenched fluorophore whose fluorescence is restored when they bind to a target nucleic acid. They are designed in such a way that the loop portion of the molecule is a probe sequence complementary to a target nucleic acid molecule. The stem is formed by the annealing of complementary arm sequences on the ends of the probe sequence. A fluorescent moiety is attached to the end of one arm and a quenching moiety is attached to the end of the other arm. The stem keeps these two moieties in close proximity to each other, causing the fluorescence of the fluorophore to be quenched by energy transfer. Since the quencher moiety is a non-fluorescent chromophore and emits the energy that it receives from the fluorophore as heat, the probe is unable to fluoresce. When the probe encounters a target molecule, it forms a hybrid that is longer and more stable than the stem and its rigidity and length preclude the simultaneous existence of the stem hybrid. Thus, the molecular beacon undergoes a spontaneous conformational reorganization that forces the stem apart, and causes the fluorophore and the quencher to move away from each other, leading to the restoration of fluorescence.

The provided method can detect the SNP using Scorpions® probes. Scorpions® probes are bi-functional molecules in which a primer is covalently linked to the probe. The molecules also contain a fluorophore and a quencher. In the absence of the target, the quencher quenches the fluorescence emitted by the fluorophore. During the Scorpions® PCR reaction, in the presence of the target, the fluorophore and the quencher separate which leads to an increase in fluorescence. The fluorescence can be detected and measured in the reaction tube. The Scorpions® primer carries a Scorpions® probe element at the 5′ end. The probe is a self-complementary stem sequence with a fluorophore at one end and a quencher at the other. The Scorpion™ primer sequence is modified at the 5′ end. It contains a PCR blocker at the start of the hairpin loop (Usually HEG monomers are added as a blocking agent). In the initial PCR cycles, the primer hybridizes to the target and extension occurs due to the action of polymerase. Scorpions® primers can be used to examine and identify point mutations by using multiple probes. Each probe can be tagged with a different fluorophore to produce different colors. In Scorpions® primers, the probe is physically coupled to the primer which means that the reaction leading to signal generation is a unimolecular one. This is in contrast to the bi-molecular collisions required by other technologies such as TaqMan® or molecular beacons. After one cycle of PCR extension completes, the newly synthesized target region will be attached to the same strand as the probe. Following the second cycle of denaturation and annealing, the probe and the target hybridize. The denaturation of the hairpin loop requires less energy than the new DNA duplex produced. Consequently, the hairpin sequence hybridizes to a part of the newly produced PCR product. This results in the separation of the fluorophore from the quencher and causes emission.

The SNP can also be detected using an allele-specific amplification primer that have secondary priming sites for universal energy-transfer-labeled primers.

The SNP can be detected using an AlphaScreen® proximity assay. AlphaScreen® generates an amplified light signal when donor and acceptor beads are brought to proximity, and this detection method can be combined with allele-specific amplification chemistry or allele-specific hybridization chemistry for allele discrimination.

3. Allele Specific Oligonucleotide Ligation

The SNP can be detected using Allele Specific Oligonucleotide Ligation. By designing oligonucleotides complementary to the target sequence, with the allele-specific base at its 3′-end or 5-′end, one can determine the genotype of the PCR amplified target sequence by determining whether an oligonucleotide complementary to the DNA sequencing adjoining the polymorphic site is ligated to the allele-specific oligonucleotide or not.

4. Invader® Method

There have been a few notable efforts to establish PCR-free genotyping methods. One such attempt is the Invader® method (Third Wave Technologies), based on a matched nucleotide-specific cleavage by a structure-specific ‘flap’ endonuclease, in the presence of an invading oligonucleotide. The combination of this reaction with a secondary reaction using fluorescence resonance energy transfer (FRET) oligonucleotide cassettes, generates a highly allele-specific signal, in a completely homogeneous and isothermal reaction. In addition, the Invader® assay's great sensitivity and excellent signal to noise ratio allow direct genotyping of genomic DNA samples without PCR. However, the amount of DNA currently required for reliable genotyping is high (50 ng range) for the analysis of a large number of SNPs. The Invader method can be combined with PCR to reduce the DNA requirement, which also makes the signal more robust.

5. Rolling Circle DNA Amplification

Another type of PCR-free genotyping is available through the combination of padlock probe ligation, and signal amplification by the rolling circle DNA amplification (RCA) process. In this assay, allele discrimination is accomplished by the specific ligation of completely matched oligonucleotides, in the same way as oligonucleotide ligation assay (OLA). The difference here is that the ligation of a padlock probe creates a circular DNA, which can be amplified by rolling circle DNA synthesis by a DNA polymerase. The high degree of signal amplification by rolling circle synthesis and the specificity of the allele-discrimination by DNA ligase, make padlock probe/RCA assay sensitive enough to be directly applied to genomic DNA. However, typical padlock probe/RCA genotyping still requires a large quantity of DNA (100 ng) per genotype, again making it less than ideal for the analysis of many SNPs. However, FRET primers (Amplifluor) can be used for signal detection in reducing the DNA requirement to a nanogram level.

6. Mass Spectroscopy

The SNP can be detected by mass spectroscopy. The principle of this method is to use mass spectrometry to detect the product of enzymatic allele-discrimination reaction directly or indirectly. Various allele discrimination chemistries such as single-base extension and its variation, allele-specific hybridization of peptide nucleic acid (PNA), Invader®, and allele-specific PCR, have all been successfully combined with the mass spectrometry detection. Combinations of single-base extension or its modifications with matrix-assisted laser desorption/ionization time-of-flight (MALDI-TOF) mass spectrometry are the most commonly used, and have been made into commercial products by companies such as Sequenom and Applied Biosystems/PerSeptive Biosystems. The advantage of the MALDI-TOF mass spectrometry-based detection is in its speed and multiplexing capability. For example, a moderate mass spectrometer capable of recording 40,000 spectra a day, can theoretically score 200,000 genotypes in a 5-plex detection format. However, their rate limiting steps are generally not in the detection process by a mass spectrometer, but are in the preceding enzymatic reactions, and post-reaction sample processing steps. In most mass spectrometry-based assays, 5-plex may be the realistic limit for multiplexing to get reliable signals, partly due to the limitations in the detectable mass range and in the sensitivity of mass discrimination. Post-reaction sample processing is more complicated than that of most other genotyping formats, as a very high purity is necessary for the samples to be analyzed by a mass spectrometer. Solid phase sample processing with ion-exchange resin is employed in Sequenom's MassArray® automated system, while miniaturized reverse phase liquid chromatography is used for Applied Biosystems/PerSeptive Biosystem's product to address this issue. Another system called the GOOD assay involves a use of chemically modified primers in the reaction, followed by an enzymatic removal of unextended primers and alkylation of the product, allowing a simplified and effective sample preparation for mass spectrometry.

Genotype accuracy due to the intrinsic nature of mass spectrometry is another advantage. The sensitivity of the instrument, the mass specificity of each reaction product, and for some type of reactions the fact that each reaction contains internal standards for calibration, all contribute to this accuracy. Mass spectrometry-based methods give little background especially when detecting the allelic discrimination reaction products directly, allowing accurate and automated genotype calling.

A different mass spectrometry-based assay has been made into a commercial product as Qiagen's MassCode® system. This assay combines allele-specific PCR with UV-cleavable ‘mass tags’, and mass spectrometry detection. Here, mass spectrometry detects the cleaved tags and not the extension products themselves. Use of these ‘mass tags’ makes highly-multiplexed detection by a relatively simple mass spectrometer possible. One the other hand, this method can be more prone to background signal at least theoretically, as the mass spectrometer does not directly detect the allele-discrimination reaction product. For example, incomplete removal of free ‘mass tag’ labeled primers before UV-cleavage can cause a false signal in this method.

Matrix-assisted laser desorption/ionization (MALDI) is a soft ionization technique used in mass spectrometry, allowing, among other things, the ionization of biomolecules (biopolymers such as proteins, peptides and sugars) which tend to be more fragile and quickly lose structure when ionized by more conventional ionization methods. A matrix is used to protect the biomolecule from being destroyed by direct laser beam and to facilitate vaporization and ionization. The matrix consists of crystallized molecules, of which the three most commonly used are 3,5-dimethoxy-4-hydroxycinnamic acid (sinapinic acid), α-cyano-4-hydroxycinnamic acid (alpha-cyano or alpha-matrix) and 2,5-dihydroxybenzoic acid (DHB). A solution of one of these molecules is made, often in a mixture of highly purified water and an organic solvent (normally acetonitrile (ACN) or ethanol). Trifluoroacetic acid (TFA) may also be added. A good example of a matrix-solution would be 20 mg/mL sinapinic acid in ACN:water:TFA (50:50:0.1). The matrix solution is generally mixed with the analyte (e.g. protein-sample). The organic solvent allows hydrophobic molecules to dissolve into the solution, while the water allows for water-soluble (hydrophilic) molecules to do the same. This solution is spotted onto a MALDI plate (usually a metal plate designed for this purpose). The solvents vaporize, leaving only the recrystallized matrix, but now with analyte molecules spread throughout the crystals. The matrix and the analyte are said to be co-crystallized in a MALDI spot. The laser is fired at the crystals in the MALDI spot. The spot absorbs the laser energy and it is thought that primarily the matrix is ionized by this event. The matrix is then thought to transfer part of its charge to the analyte molecules (e.g. protein), thus ionizing them while still protecting them from the disruptive energy of the laser. Ions observed after this process are quasimolecular ions that are ionized by the addition of a proton to [M+H]+, or other cation such as sodium ion [M+Na]+, or the removal of a proton [M−H]− for example. MALDI generally produces singly-charged ions, but multiply charged ions ([M+nH]n+) can also be observed, usually in function of the matrix used and/or of the laser intensity, voltage.

7. Sequencing

The SNP can be detected using gene sequencing. Sequencing is the procedure of choice for SNP discovery. The most common forms of sequencing are based on primer extension using either a) dye-primers and unlabeled terminators or b) unlabeled primers and dye-terminators. The products of the reaction are then separated using electrophoresis using either capillary electrophoresis or slab gels.

Pyrosequencing employs an elegant cascade of enzymatic reactions to detect nucleotide incorporation during DNA synthesis. When a nucleotide is incorporated at the 3′-end by DNA polymerase, a pyrophosphate is released that is immediately converted to ATP by ATP sulfurylase. This ATP causes the oxidization of luciferin by luciferase, which is detected as a light signal. Pyrosequencing was initially developed as a DNA sequencing method, with a chemistry completely different from the Sanger dideoxynucleotide method. It is also a unique homogeneous sequencing method with no electrophoresis. Its capability to read flanking sequences as well as the SNP position itself, and its high specificity (i.e., non-specific binding will not generate a false signal) make it an accurate and attractive SNP genotyping method. In this method, alleles can be called by analyzing the individual sample itself, without comparing its signal to that of other samples or controls. This makes pyrosequencing suitable for fully automated genotype calling, an important component of high throughput analyses. A 96-well medium throughput machine and a fully automated 384-well format high-throughput machine, are available from Pyrosequencing AB (Uppsala, Sweden) for this method, and the latter has capacity to score high thousands to low tens of thousands of genotypes a day. Pyrosequencing can be done in a duplex or a triplex format at least for some SNP combinations.

### II. SIRT5ₚᵣₒₘ₂ SNP Probes and Primers

The neighboring sequence to the polymorphic site can be used to design SNP detection reagents such as oligonucleotide probes and primers. One embodiment provides compositions including primers and probes capable of interacting with the disclosed nucleic acids containing SIRT5ₚᵣₒₘ₂. In certain embodiments the primers are used to support DNA amplification reactions. Typically the primers will be capable of being extended in a sequence specific manner. Exemplary primers or probes are at least 10, 11, 12, 13, 14, 15, 16, 17, 18, 19 or 20 contiguous nucleotides of SEQ ID NO:1, SEQ ID NO:2 or a complement thereof. In one embodiment the primer or probe spans the cystidine at nucleotide position 27 of SEQ ID NO:1.

Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

Typically, an oligonucleotide probe or primer will comprise a region of nucleic acid sequence that hybridizes to at least about 8, more preferably at least about 10 to about 15, typically about 20 to about 40 consecutive nucleotides of a target nucleic acid (i.e., will hybridize to a contiguous sequence of the target nucleic acid). In one embodiment the probe is at least 14 nucleotides. Oligonucleotides that exhibit differential or selected binding to a polymorphic site may readily be designed by one of ordinary skill in the art. For example, an oligonucleotide that is perfectly complementary to a sequence that encompasses a polymorphic site will hybridize to a nucleic acid comprising that sequence as opposed to a nucleic acid comprising an alternate polymorphic variant.

### III. SIRT5ₚᵣₒₘ₂ SNP Detection Kits

Detection reagents can be developed and used to detect the disclosed SIRT5ₚᵣₒₘ₂ SNP, and the detection reagents can be readily incorporated into a kit or system format. SNP detection kits and systems, include but are not limited to, a packaged probe and primer sets (e.g., TaqMan® probe/primer sets), arrays/microarrays of nucleic acid molecules, and beads that contain one or more probes, primers, or other detection reagents for detecting the disclosed SNP. The kits/systems can optionally include various electronic hardware components; for example, arrays (“DNA chips”) and microfluidic systems (“lab-on-a-chip” systems) provided by various manufacturers typically include hardware components. Other kits/systems (e.g., probe/primer sets) may not include electronic hardware components, but may include one or more SNP detection reagents (along with, optionally, other biochemical reagents) packaged in one or more containers.

In some embodiments, a SNP detection kit typically contains one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a SNP-containing nucleic acid molecule. A kit may further contain means for determining the amount of a target nucleic acid, and means for comparing the amount with a standard, and can comprise instructions for using the kit to detect the SNP-containing nucleic acid molecule of interest. In one embodiment, kits are provided which contain the necessary reagents to carry out one or more assays to detect the disclosed SNPs. In an exemplary embodiment, SNP detection kits/systems are in the form of nucleic acid arrays, or compartmentalized kits, including microfluidic/lab-on-a-chip systems.

In other embodiments, SNP detection kits may contain, for example, one or more probes, or pairs of probes, that hybridize to a nucleic acid molecule at or near each target SNP position. In some kits, the allele-specific probes are immobilized to a substrate such as an array or bead. The terms “arrays”, “microarrays”, and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. Any number of probes, such as allele-specific probes, may be implemented in an array, and each probe or pair of probes can hybridize to a different SNP position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized. Probes can be attached to a solid support in an ordered, addressable array.

A microarray can be composed of a large number of unique, single-stranded polynucleotides, usually either synthetic antisense polynucleotides or fragments of cDNAs, fixed to a solid support. Typical polynucleotides are about 6-60 nucleotides in length, or about 15-30 nucleotides in length, or about 18-25 nucleotides in length. For certain types of microarrays or other detection kits/systems, it may be preferable to use oligonucleotides that are only about 7-20 nucleotides in length. In other types of arrays, such as arrays used in conjunction with chemi luminescent detection technology, exemplary probe lengths can be, for example, about 15-80 nucleotides in length, or about 50-70 nucleotides in length, or about 55-65 nucleotides in length, or about 60 nucleotides in length. The microarray or detection kit can contain polynucleotides that cover the known 5′ or 3′ sequence of a gene/transcript or target SNP site, sequential polynucleotides that cover the full-length sequence of a gene/transcript; or unique polynucleotides selected from particular are as along the length of a target gene/transcript sequence. Polynucleotides used in the microarray or detection kit can be specific to a SNP or SNPs of interest (e.g., specific to a particular SNP allele at a target SNP site, or specific to particular SNP alleles at multiple different SNP sites).

Hybridization assays based on polynucleotide arrays rely on the differences in hybridization stability of the probes to perfectly matched and mismatched target sequence variants. For SNP genotyping, it is generally preferable that stringency conditions used in hybridization assays are high enough such that nucleic acid molecules that differ from one another at as little as a single SNP position can be differentiated. Such high stringency conditions may be preferable when using, for example, nucleic acid arrays of allele-specific probes for SNP detection. In some embodiments, the arrays are used in conjunction with chemiluminescent detection technology.

A polynucleotide probe can be synthesized on the surface of the substrate by using a chemical coupling procedure and an inkjet application apparatus as known in the art. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures.

Methods for using such arrays or other kits/systems, to identify SNPs and haplotypes disclosed herein in a test sample are provided. Such methods typically involve incubating a test sample of nucleic acids with an array having one or more probes corresponding to at least one SNP position of the present invention, and assaying for binding of a nucleic acid from the test sample with one or more of the probes. Conditions for incubating a SNP detection reagent (or a kit/system that employs one or more such SNP detection reagents) with a test sample vary. Incubation conditions depend on such factors as the format employed in the assay, the detection methods employed, and the type and nature of the detection reagents used in the assay.

In other embodiments, a SNP detection kit/system can include components that are used to prepare nucleic acids from a test sample for the subsequent amplification and/or detection of a SNP-containing nucleic acid molecule. Such sample preparation components can be used to produce nucleic acid extracts (including DNA and/or RNA), proteins or membrane extracts from any bodily fluids (such as blood, serum, plasma, urine, saliva, phlegm, gastric juices, semen, tears, sweat, etc.), skin, hair, cells (especially nucleated cells), biopsies, buccal swabs or tissue specimens.

Another form of kit is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other SNP detection reagent for detecting one or more of the disclosed SNPs, one or more containers which contain wash reagents (such as phosphate buffered saline, Tris-buffers, etc.), and one or more containers which contain the reagents used to reveal the presence of the bound probe or other SNP detection reagents. The kit can optionally further include compartments and/or reagents for, for example, nucleic acid amplification or other enzymatic reactions such as primer extension reactions, hybridization, ligation, electrophoresis (e.g., capillary electrophoresis), mass spectrometry, and/or laser-induced fluorescent detection. The kit may also include instructions for using the kit.

Microfluidic devices may also be used for analyzing SNPs. Such systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect one or more of the disclosed SNPs. For genotyping SNPs, an exemplary microfluidic system may integrate, for example, nucleic acid amplification, primer extension, capillary electrophoresis, and a detection method such as laser induced fluorescence detection.

### EXAMPLES

### Example 1

### Molecular Aging is Conserved Across Cohorts and Brain Areas

Materials and Methods

Cohorts and Microarrays

Two previously described microarray datasets were used: Cohort 1 (Erraji-Benchekroun, et al., Biol Psychiatry, 57:549-58 (2005)) [39 subjects; ages 14-79; prefrontal cortex (PFC) Brodmann area 9 (BA9) and 47 (BA47) samples] and Cohort 2 (Sibille, et al., Am J Psychiatry, 166:1011-24 (2009)) [36 subjects, ages 23-71; anterior cingulate cortex (ACC) and amygdale (AMY) samples]. Subject characteristics, dissection protocols, and array controls were described in (Erraji-Benchekroun, et al., Biol Psychiatry, 57:549-58 (2005); Sibille, et al., Am J Psychiatry, 166:1011-24 (2009)) and are summarized in Table 1, shown below.

TABLE 1
Summary of Cohorts

| | Cohort 1 | | Cohort 2 | |
|---|---|---|---|---|
| Number of Subjects | 37 | | 39 | |
| Age Range | 23-71 | | 14-79 | |
| Exclusion Criteria | Neurodegenerative disease, | | Neurodegenerative disease, | |
| | schizophrenia, bipolar disorder, | | schizophrenia, bipolar disorder, | |
| | prolonged post-mortem interval/ | | prolonged post-mortem interval/ | |
| | agonal time, illicit drugs. | | agonal time, illicit drugs. | |
| Included Diagnoses | Major Depression | | Major Depression | |
| | (50% of Subjects) | | (50% of Subjects) | |
| Microarray Platform | Affymetrix U133 Plus | | Affymetrix U133A | |
| Number of Probesets/Genes in Data Sets | | | | |
| Genes (Expressed Genes) | 21,115 (15,522) | | 13,795 (13,520) | |
| Probesets (Expressed Probesets) | 54,715 (35,122) | | 22,177 (21,961) | |
| Brain Areas | ACC (BA25) | AMY | PFC (BA9) | PFC |
| | | | | (BA47) |
| Aging Significant Probesets (% of total) | | | | |
| α | | | | |
| 0.001 | 1309 (3.9%) | 814 (2.3%) | 1972 (9.0%) | 1106 (4.6%) |
| 0.01 | 4443 (13.3%) | 2820 (8%) | 4240 (19.3%) | 2801 (12.8%) |
| Aging Estimated False Discovery Rate | | | | |
| (FDR) | | | | |
| α | | | | |
| 0.001 | 2.8% | 4.3% | 1.2% | 2.0% |
| 0.01 | 8.0% | 12.4% | 5.2% | 7.8% |
| Major Depression Significant Probesets | | | | |
| (fold change < aging at same α) | | | | |
| α | | | | |
| 0.001 | 4 (330X) | 4 (200X) | 2 (986X) | 6 (184X) |
| 0.01 | 93 (48X) | 69 (40X) | 87 (49X) | 68 (41X) |
| Major Depression FDR | | | | |
| α | | | | |
| 0.001 | >100% | >100% | >100% | >100% |
| 0.01 | >100% | >100% | >100% | >100% |

All subjects were free of age-related neurological diseases at time of death according to medical records and pathologist examination of brain tissue. GC-RMA-extracted data from Affymetrix HU133A (cohort 1) and HU133Plus2.0 (cohort 2) arrays were used. Control variables included technical measures (chip quality controls, RNA integrity, postmortem interval) and subject characteristics (race, gender, and mode of death).

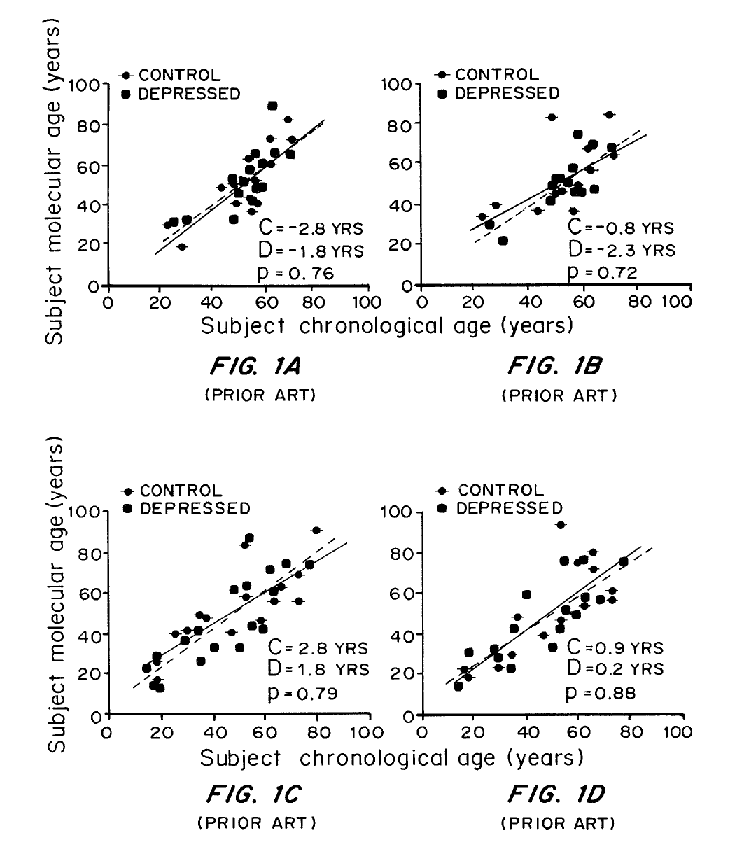

Importantly, both cohorts included subjects diagnosed with major depression (Table 1). It has previously been shown (and confirmed here) that the gene expression correlates of depression were of greatly reduced scope compared to the effects of aging. Specifically, Table 1 shows for both cohorts that the effect sizes of aging are between 184 and 986 times greater than the effect sizes of major depression at the same significance cutoff of p<0.001 (aging: 814-1972 transcripts per brain area, depression: 2-6 transcripts per brain area) and 40-50 times greater at the p<0.01 cutoff, and that major depression effects do not survive Benjamini-Hochberg control for multiple testing. Moreover, as previously described (Erraji-Benchekroun, et al., Biol Psychiatry, 57:549-58 (2005), major depression was not associated with deviations in molecular ages (FIG. 1A-D). So since human brain samples are a limited resource and as the effects of depression are of limited scope and do not associate with altered rates of molecular aging, these subjects were included in the current analysis in order to increase analytical power.

Cohort 1 (PFC BA9&47) Description Adapted from Erraji-Benchekroun et al. 2005

Subjects

Samples from 39 subjects, ranging from 14 to 79 years of age (44+/−20 years, Mean+/−SD) were obtained from the brain collection of the Human Neurobiology Core of the Conte Center for the Neuroscience of Mental Disorders, the New York State Psychiatric Institute. All cases were clinically free of neurologic disease, as determined by psychological autopsy and neuropathologic examination, including thioflavine S or immunohistochemical stains on fixed tissue for senile plaques and neurofibrillary tangles. Varying degrees of atherosclerosis were present in subjects aged 45 or older, and several specimens included foci of encephalomalacia, as expected during normal aging. Several subjects contained senile plaques or neurofibrillary tangles, but never in sufficient numbers to suggest a diagnosis of Alzheimer's disease. No other significant abnormalities were observed. All subjects died rapidly, 20 of which committed suicide (psychological autopsies indicated that 17 of them had a lifetime diagnosis of major depression) and 19 died of causes other than suicide. An independent study assessed the effect of suicide and depression on gene expression and within current analytical limits, found no evidence for molecular differences that correlated with depression and suicide. Using body fluids and brain tissue, a toxicologic screen was carried out for the presence of psychotropic or illegal drugs. All samples were psychotropic medication-free with minimal other drug exposures. Caucasians repiesented 71%, African Americans 8%, Hispanics 18%, and Asians 2% of the sample. Average postmortem interval and brain pH were 17±1 and 6.53±0.21, respectively. As a group, male subjects (n=30) did not differ significantly from female subjects (n=9) on age, race, postmortem delay, or brain pH. No interaction among experimental, demographic, and clinical parameters and age were found. Hence, all samples were combined for this aging study. RNA extraction, microarray samples preparation, and quality control were performed according to the manufacturer protocol (http://www.affymetrix.com). Samples were hybridized to Affymetrix U133A microarrays.

Cohort 2 (AMY and ACC). Adapted from Sibille et al. 2009 Subjects

39 all male subject (ages 23-71) brain samples were obtained during autopsies conducted at the Allegheny County Medical Examiner's Office. Subjects with advanced disease stages (i.e., cancer, neurodegenerative disorders) and prolonged postmortem interval PMI (>28 hrs) were excluded. All subjects were white Caucasian and were selected for rapid modes of death and short agonal phases, to limit the influence of agonal factors on RNA quality and pH. Toxicological screens on peripheral fluids identified the presence of at least one antidepressant in 5 subjects, including four different tricyclics, one selective serotonin reuptake inhibitor and one weak dopamine reuptake inhibitor.

Data Extraction, Normalization, and Creation of Best-Fit Age-Trajectory Equations

Log₂-transformed probeset signal intensities were extracted and normalized with the Robust Multi-array Average (GC-RMA) algorithm for each brain area for both datasets. Probesets were considered present if they had expression levels greater than 25 in at least 2 datasets in order to preserve area/cohort specificities if present. Expression values were then converted for comparability by simple division to be a fraction of their mean value of all expression values for their probeset in each brain area separately. Then, for each probeset, a separate equation was generated for linear, log, exponential, and power fits of expression versus subject age and the best-fit line (highest regression coefficient) was chosen for each probeset, creating a unique age-regression equation per probeset. Lastly, expression values were converted a second time by simple division to be a percentage of their expression at age 20 yrs (calculated by solving their equation for this value), which was set to 100% expression. Equations were re-calculated using this value. This created equations and expression values directly comparable across datasets that are a percentage of expression at age 20 yrs, which was set to 100% expression, without transforming or altering original expression. P-values for age-trajectory equations were calculated by converting regression coefficients of equations taking into account the number of subjects in each brain area.

Best-Fit Age-Regression

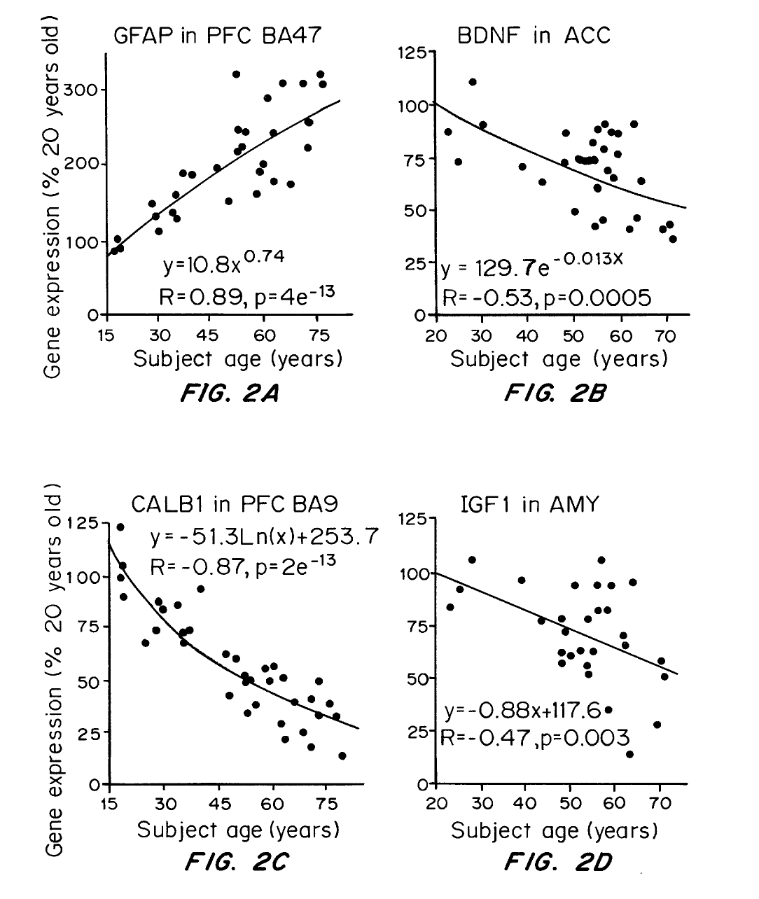

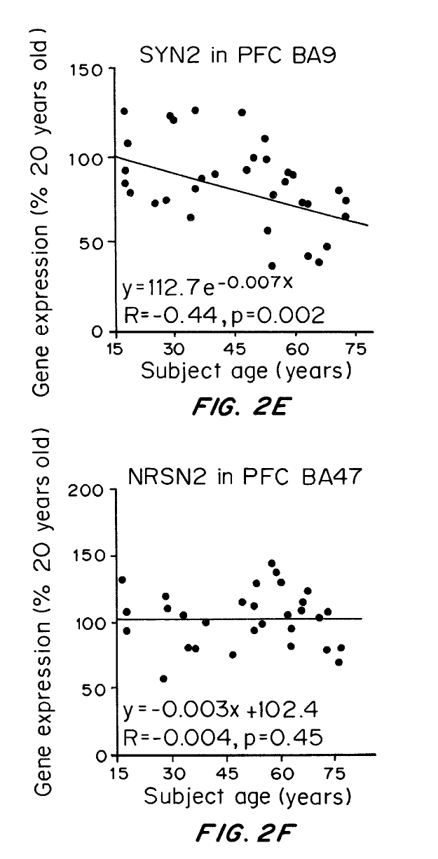

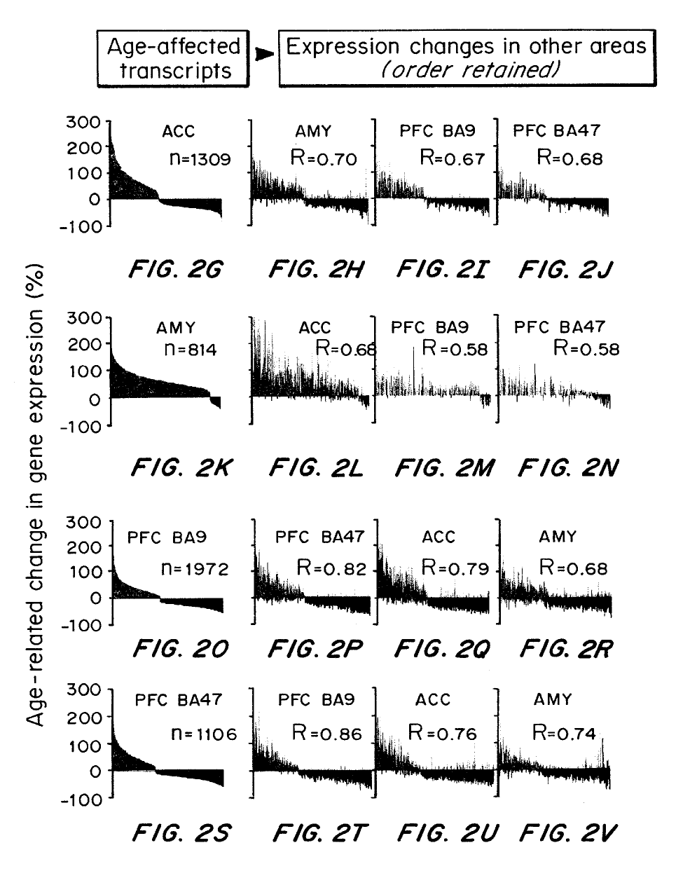

For congruence with the progressive pattern of structural (decreasing grey matter) and functional (cognitive decline) brain aging changes (Brickman, et al., Biol Psychiatry, 60:444-53 (2006); Resnick, et al., J Neurosci., 23:3295-301 (2003)), best-fit age-regression coefficients were used to determine significance of age-related gene transcript changes across subjects (FIG. 2A-F, Tables 1, 2, shown below).

TABLE 2
Percentage of each best-fit equation type by brain area.

| | | | | Log |
|---|---|---|---|---|
| | Linear (%) | Exponential (%) | Power (%) | (%) |
| ACC | 36 | 21 | 30 | 13 |
| AMY | 12 | 23 | 60 | 5 |
| BA9 | 34 | 27 | 16 | 22 |
| BA47 | 29 | 22 | 20 | 29 |

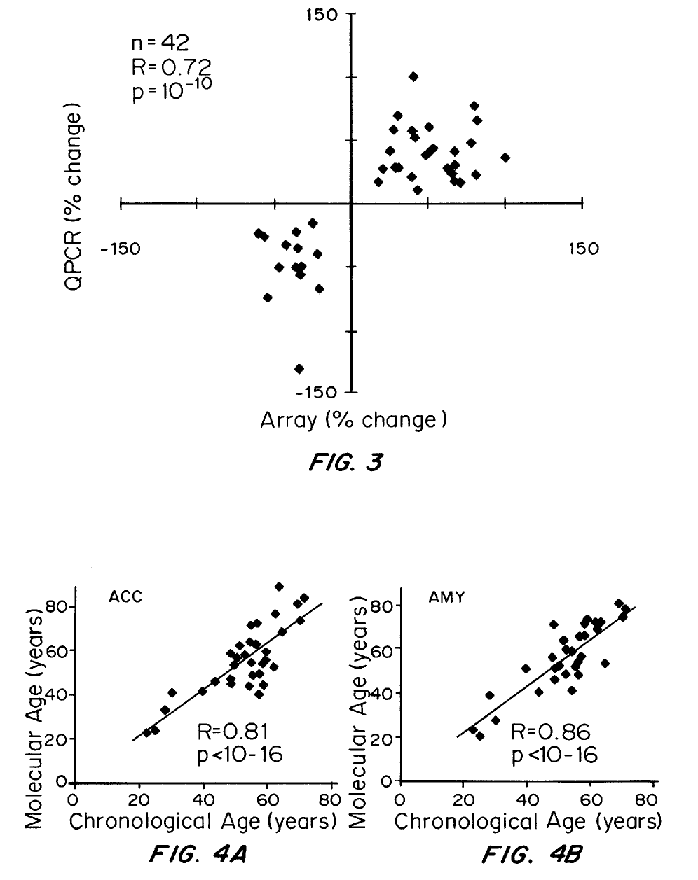

For each transcript, equations were generated for linear, log, exponential, and power fits of expression level versus chronological age and the most significant (best-fit) equation was selected (p-values derived from correlation R-values). False discovery rates (FDR) were estimated using Benjamini-Hochberg methodology (Benjamini, et al., J Royal Stat Soc Series B Methodol, 57:289-300 (1995). QPCR validation for 42 array-defined age-regulated genes are described in (Erraji-Benchekroun, et al., Biol Psychiatry, 57:549-58 (2005); Sibille, et al., Am J Psychiatry, 166:1011-24 (2009)) and in FIG. 3.

Molecular Ages

Individual predicted molecular ages were calculated for all age-regulated genes using a leave one out approach within ACC or AMY (FIG. 4A-B), as previously described (Erraji-Benchekroun, et al., Biol Psychiatry, 57:549-58 (2005)). Briefly, to describe each sample individually in the general aging trend, a one-number-summary (“Molecular age”) was devised for each sample, describing the “predicted age” of the sample when removed from the analysis. For each sample, the remaining database was analyzed for age-related genes using the same correlation-based methods described above, controlling the FDR at 0.05. For each selected gene, a best-fit regression analysis with age was performed, and the age for the held-out sample was predicted using the resulting function. Extreme outlier molecular ages (+/−10 standard deviations from average chronological age) were removed. The resulting gene-wise predicted values were averaged per sample and used to described the predicted molecular age of each subject.

SIRt5ₚᵣₒₘ₂ (rs9382222) Effects on Molecular Age

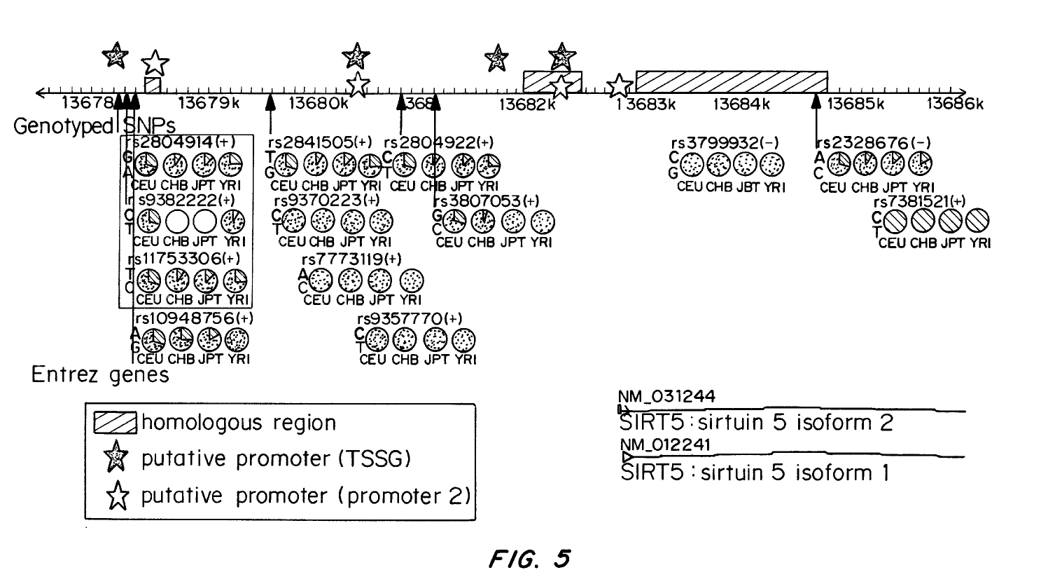

SIRT5ₚᵣₒₘ₂ is located in a mouse/human conserved region predicted by two separate programs to contain a promoter, TSSG CGG Nucleotide Sequence Analysis (http://genomic.sanger.ac.uk/gf/gf.html) and Promoter 2.0 (http://www.cbs.dtu.dk/services/Promoter/) (FIG. 5). Cohort 2 subjects were genotyped by sequencing of polymerase chain reaction (PCR) amplified segments of genomic DNA obtained from brain samples. Subjects were 50% C/C and 37% C/T in agreement with Hap-map (www.hapmap.org) published frequencies for CEU subjects (Table 3, shown below).

TABLE 3
Candidate Longevity SNP Subject Genotypes and Comparison with
Reported Frequencies.

| | | | | Hapmap (CEU)* |
|---|---|---|---|---|
| | refsnp | | | or Ensembl** |
| | (common | | | Published |
| Gene | name) | Allele | Allele frequencies | frequencies |
| HTR- | rs6296 | G/G, G/C, | 0.44, 0.47, 0.0 | 0.40, 0.52, 0.08* |
| 1B | (G861C) | C/C | | |
| SIRT5 | rs2804914 | G/G, G/A, | 0.44, 0.55, 0.0 | 0.46, 0.46, 0.09* |
| | (prom1) | A/A | | |
| | rs938222 | C/C, C/T, | 0.50, 0.37, 0.13 | 0.43, 0.46, 0.10* |
| | (prom2) | T/T | | |
| | rs11753306 | T/T, T/C, | 0.42, 0.58, 0.0 | 0.47, 0.45, 0.08* |
| | (prom3) | C/C | | |
| Klotho | rs9536314 | T/T, T/G, | 0.76, 0.24, 0.0 | 0.79, 0.21, 0.0 ** |
| | (KL-VS) | G/G | | |

Rare T/T subjects were excluded from analysis because of lack of power. Genotypic differences in all gene transcript levels were calculated using two-tailed Students t-tests in middle-aged cross-sectional groups rigorously matched for chronological age, C/C (n=12, average age=52.1 years, range=49-63 years) C/T (n=11, average age 52.7 years, range 48-64 years). Similarly significant (although ˜10% fewer affected genes) results were obtained using the alternative approach of including all subjects and controlling for age and other parameters by ANOVA.

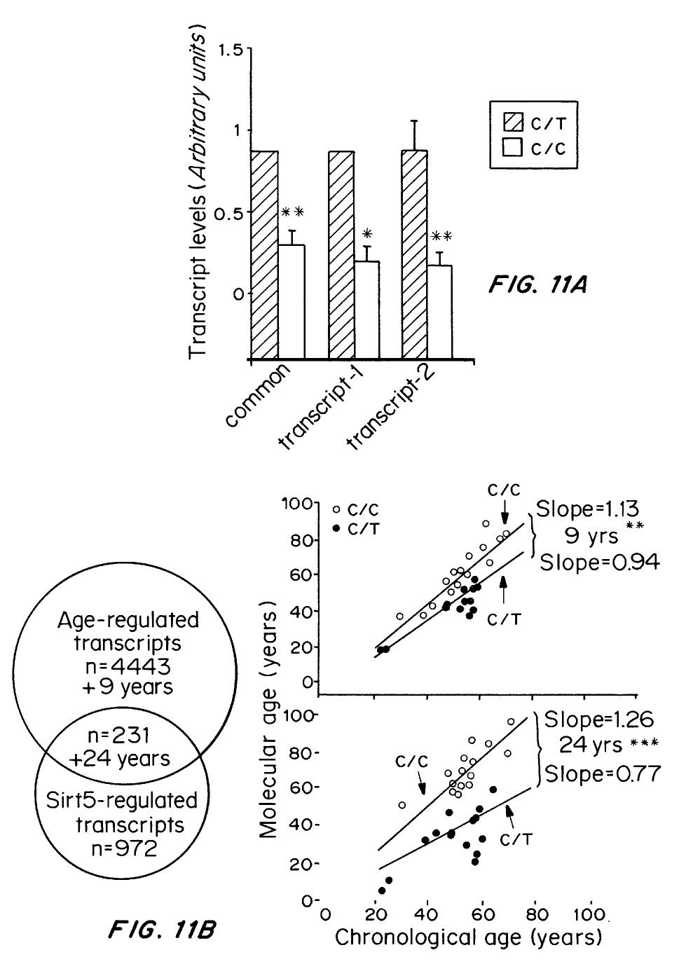

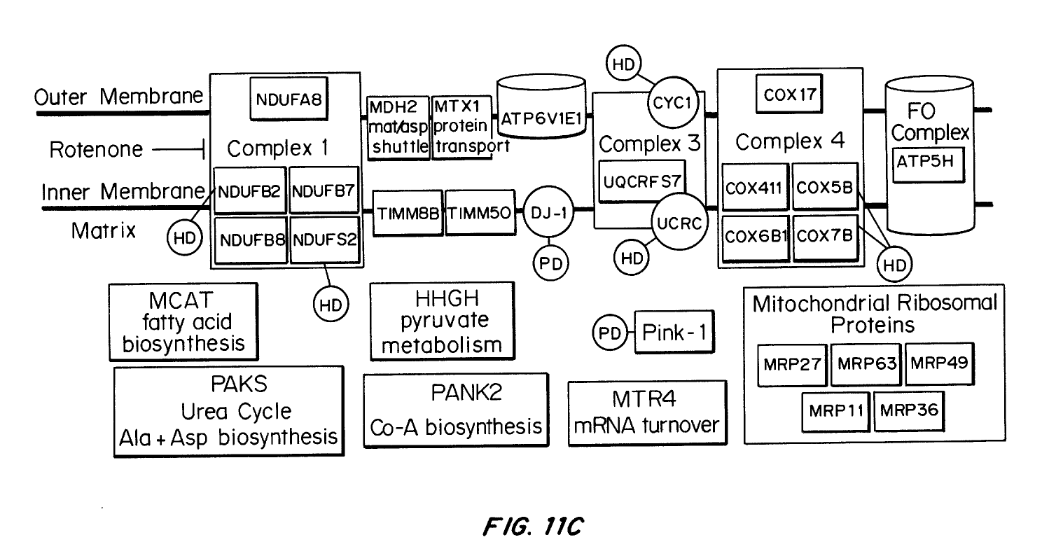

To assess snp-based group differences, molecular ages were subtracted from chronological ages to assess deviations of molecular from chronological age, thus removing the effect of chronological age. Two-tailed t-tests were performed to obtain p-values associated with difference in total molecular age between genotype-defined groups. A parallel analysis using an ANOVA model yielded similar and significant results, although slightly less robust. This analysis was also performed using only age x snp effect intersection transcripts (FIG. 11B). These transcripts are referred to here as ‘intersection transcripts’.

Results

Two previously described microarray datasets were used to investigate the extent and conservation of altered gene expression with age in the human brain (See Methods): Cohort 1 [39 subjects; ages 14-79; prefrontal cortex (PFC) Brodmann area 9 (BA9) and 47 (BA47) samples] and Cohort 2 [36 subjects, ages 23-71; anterior cingulate cortex (ACC) and amygdala samples]. At p<0.001, 814-1972 transcripts were age-regulated in each brain area with 1-4% estimated FDR (Table 1). Array data were previously validated by high correlation with independent quantitative PCR (qPCR) results (n=42 genes, R=0.72, p=10⁻¹⁰, FIG. 3) and by known age-regulated genes changing in predicted directions, including up-regulated reactive gliosis markers (GFAP), and down-regulated growth factors (BDNF and IGF-1), synaptic markers (SYN2) and calcium homeostasis genes (CALB-1) (Berchtold, et al., Proc Natl Acad Sci USA, 105:15605-10 (2008); Lu, et al., Nature, 429:883-91 (2004) (FIG. 2F). Expression changes did not reflect age-related changes in cell number, as many neuronal-specific transcripts were unchanged with age [NRSN2 (Nakanishi, et al., Brain Res, 1081:1-8 (2006); FIG. 2F], consistent with stereological studies demonstrating minimal neuronal loss during normal aging (Morrison, et al., Science, 278:412-9 (1997)).

Molecular aging was remarkably conserved across cohorts and brain areas (p<10⁻¹⁰, FIGS. 2G-V). Gross area-specific differences were only observed in amygdala, with fewer age down-regulated transcripts (FIG. 2G-V, n=87) compared to cortical areas (n=684-1133). It has previously been shown that down and up-regulated changes are predominantly of neuronal and glial origin respectively (Erraji-Benchekroun, et al., Biol Psychiatry, 57:549-58 (2005); Sibille, et al., J Neurosci Methods, 167:198-206 (2008) (Table 4, shown below).

TABLE 4
Average magnitude (age 70-age 20) and cellular origin of age-
regulated expression changes by up and down-regulated (FIG. 2G-V).

| | Increased with Age (p < 0.001) | | | | | Decreased with Age (p < 0.001) | | | | |
|---|---|---|---|---|---|---|---|---|---|---|
| | N | M | N | G | B | N | M | N | G | B |
| | (% of total) | (%) | (%) | (%) | (%) | (% of total) | (%) | (%) | (%) | (%) |
| ACC | 582 (44.5) | +95.1 | 3.0 | 73.7 | 23.4 | 726 (55.5) | −31.8 | 57.9 | 3.6 | 38.7 |
| AMY | 726 (88.6) | +74.8 | 6.1 | 47.0 | 47.0 | 87 (10.7) | −33.1 | 32.2 | 5.8 | 63.2 |
| BA9 | 838 (42.5) | +47.7 | 3.4 | 66.8 | 17.1 | 1133 (57.5) | −29.5 | 59.0 | 8.5 | 32.5 |
| BA47 | 420 (37.9) | +59.7 | 12.4 | 49.1 | 38.8 | 684 (61.8) | −31.9 | 69.9 | 6.6 | 23.7 |

(n) number,
(m) average magnitude,
(N) neuronal,
(G) Glial,
(B) expressed in both neurons and glia.

Thus the fewer observed downregulated neuronally-enriched changes still correlated with changes in other brain areas, but were “noisier” (higher p-values, Tables 4 and 5-6, shown below), consistent with structural MRI studies reporting robust cortical and more variable amygdala age-related grey matter losses (Good, et al., Neuroimage, 14:21-36 (2001)).

TABLE 5
Age-related expression changes (FIGS. 2G-V) compared across brain areas.

| | ACC | | AMY | | BA9 | | BA47 | |
|---|---|---|---|---|---|---|---|---|
| | Same (%) | Opposite (%) | Same (%) | Opposite (%) | Same (%) | Opposite (%) | Same (%) | Opposite (%) |
| ACC | — | — | 89.1 | 10.9 | 90 | 11 | 88.6 | 12.4 |
| AMY | 89.9 | 11.1 | — | — | 90.5 | 10.5 | 84.1 | 15.9 |
| BA9 | 87.4 | 14.6 | 79.4 | 20.6 | — | — | 88.3 | 14.7 |
| BA47 | 88.0 | 12.0 | 84.1 | 15.9 | 94.1 | 5.9 | — | — |

“Same”, expression changed in the same direction;
“Opposite”, expression changed in the opposite direction.

TABLE 6
Age-related expression changes compared across areas (FIGS. 2G-V).

| | ACC (%) | | | | AMY (%) | | | | BA9 (%) | | | | BA47 (%) | | | |
|---|---|---|---|---|---|---|---|---|---|---|---|---|---|---|---|---|
| | S-S | S-NS | O-S | O-NS | S-S | S-NS | O-S | O-NS | S-S | S-NS | O-S | O-NS | S-S | S-NS | O-S | O-NS |
| ACC | — | — | — | — | 43.0 | 46.1 | 0.7 | 8.7 | 75.1 | 14.9 | 1.0 | 9.0 | 57.4 | 31.2 | 1.0 | 10.4 |
| AMY | 60.2 | 29.7 | 0.9 | 9.8 | — | — | — | — | 67.3 | 23.2 | 3.0 | 6.5 | 47.0 | 37.1 | 2.7 | 13.8 |
| BA9 | 58.0 | 29.5 | 2.1 | 10.4 | 31.8 | 47.6 | 2.6 | 18.0 | — | — | — | — | 65.4 | 22.9 | 3.0 | 8.7 |
| BA47 | 54.1 | 33.9 | 1.2 | 10.8 | 34.8 | 50.5 | 1.4 | 13.3 | 77.9 | 16.2 | 0.5 | 5.4 | — | — | — | — |

Analyzed by percentage of transcripts in same (S) or opposite (O) directions across two brain areas and (—) whether they were significant p < 0.05 (S) or non-significant (NS).

### Example 2

### Age-Related Biosignature Predicts Chronological Age, Contains Development- and Neurological Disease-Related Genes, and is Potentially Regulated by Cell-Cycle and Neurotransmitter-Modulatory Drugs

Materials and Methods

Age-Related Biosignatures

Genes were included in the cross-area biosignature if they displayed age-regression p<0.01 with age in ¾ brain areas and p<0.05 in the fourth, and if directions of age-regulated changes were concordant in all brain areas. Notably, all but one gene that met the first criteria did not pass the second (HTR2A—both probesets increased with age in amygdala but decreased in cortical areas). If more than one probeset per gene met both criteria, the probeset with the lowest p-value across areas was selected to avoid any gene having a greater weighted influence on molecular age. For ACC and amygdala-specific biosignatures, genes were selected if they had age-regression p<0.01 in those areas. Cross-area biosignature genes, cross-area equations, regression R-values, p-values, and magnitude of expression changes are available on-line (www.sibille.pittedu/data.html).

Real-Time Quantitative PCR (qPCR)

qPCR was performed as previously described (Sibille, et al., Am J Psychiatry, 166:1011-24 (2009)). Results were calculated as the geometric mean of relative intensities compared to three internal controls (actin, glyceraldehyde-3-phosphate dehydrogenase and cyclophilin).

Transcriptome Functional Analyses

Analyses were performed using Ingenuity® version 7.0. and the connectivity map (C-MAP), as described in the respective websites [http://www.ingenuity.com/; http://www.broadinstitute.org/cmap, (Lamb, et al., Sciene, 313:1929-35 (2006)] and in the supplements.

Cross-Sectional Brain Area Comparisons

Transcripts with age-regression p<0.001 were selected for each brain area, and regression equations were solved for percentile expression changes between 20 and 70 years of age. Directed Pearson correlations (Oh, et al., Neurobiol Aging, (2009)) were performed by correlating these expression changes with transcript levels for the same genes in the other three brain areas.

Ingenuity Analysis of the Biosignature

356 age-biomarkers were used for gene network and associated functional analysis. A summary of functional and network analysis is shown below. The top 5 identified gene networks (p<e⁻³⁵) encompassed most known age-related biological functions (Signaling, immune response, vascular function, cell death, DNA repair and protein modification; Networks 1-5) and confirmed the substantial overlap between age and disease-related genes (Genetic, neurological and psychiatric disorders; Networks 3-4; Table S7).

Results

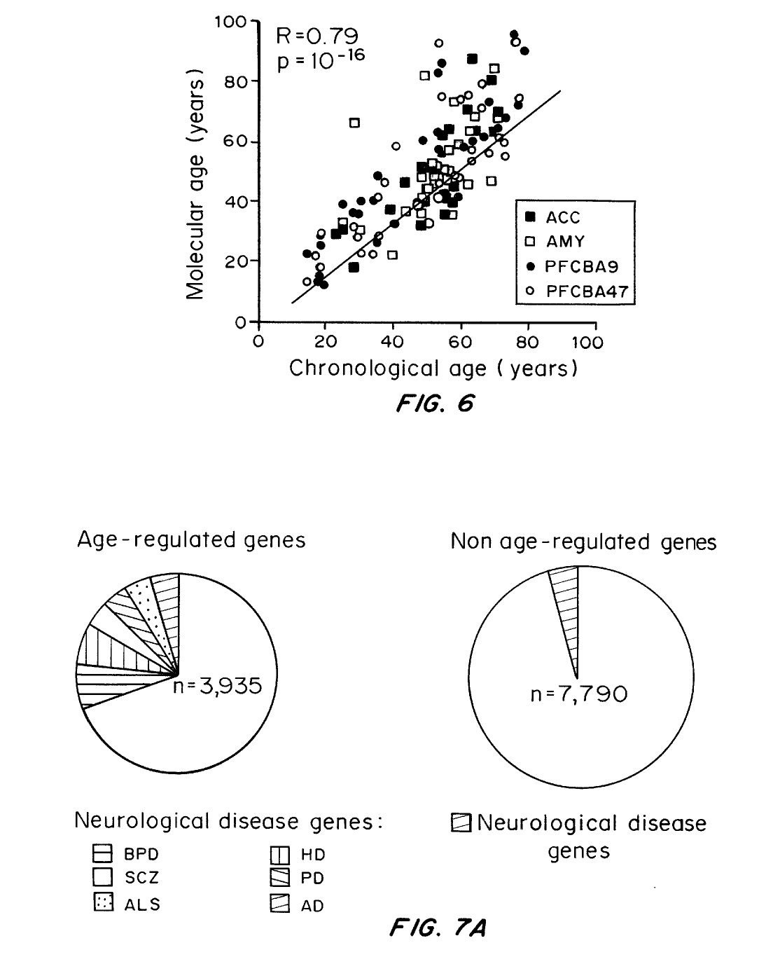

To assess cross-sectional rates of molecular aging, a brain- and age-related biosignature was developed, based on conserved changes across areas (n=356-genes). Transcript levels were converted into “molecular ages” using cross-area age-regression equations, which were averaged to generate a single molecular age per subject per brain area, using a leave-one-out approach to avoid circularity. The biosignature was highly predictive of subject age (p<10⁻¹⁶, FIG. 6), confirming its utility as a quantitative assay and the cross-area robustness of age-related transcript changes.

Using large-scale hand-annotated literature information, Ingenuity® biological pathway analysis identified the expected categories of known age-related changes in the biosignature (cell morphology, signaling, immune response, vascular function, cell death, DNA repair and protein modification) (Tables 7 and 8, shown below).

TABLE 7
Summary of Ingenuity Analysis of the biosignature
Top Networks

| ID | Associated Network Functions | Score |
|---|---|---|
| 1 | Cell-To-Cell Signaling and Interaction, Cell-mediated Immune | 39 |
| | Response, Hematological System Development | |
| | and Function | |
| 2 | Nervous System Development and Function, Tissue | 37 |
| | Development, DNA Replication, Recombination, | |
| | and Repair | |
| 3 | Genetic Disorder, Neurological Disease, Cell Death | 35 |
| 4 | Genetic Disorder, Neurological Disease, Psychological | 35 |
| | Disorders | |
| 5 | Post-Translational Modification, Protein Folding, Nervous | 35 |
| | System Development and Function | |

Top Bio Functions

| | | |
|---|---|---|
| Name | p-value | # Molecules |

Diseases and Disorders

| | | |
|---|---|---|
| Genetic Disorder | 1.21E−18-2.70E−02 | 152 |
| Neurological Disease | 1.21E−18-2.70E−02 | 115 |
| Psychological Disorders | 2.49E−09-2.30E−02 | 34 |
| Cancer | 8.36E−07-2.70E−02 | 127 |
| Dermatological Diseases and | 1.40E−05-2.70E−02 | 20 |
| Conditions | | |

Molecular and Cellular Functions

| | | |
|---|---|---|
| Cell Morphology | 5.20E−07-2.70E−02 | 57 |
| Cell Death | 1.40E−05-2.70E−02 | 84 |
| Cellular Assembly and Organization | 5.37E−05-2.70E−02 | 60 |
| Cell Signaling | 3.50E−04-2.70E−02 | 40 |
| Cellular Development | 3.60E−04-2.70E−02 | 84 |

Physiological System Development and Function

| | | |
|---|---|---|
| Nervous System Development | 5.20E−07-2.70E−02 | 70 |
| and Function | | |
| Behavior | 2.75E−05-2.70E−02 | 28 |
| Tissue Development | 7.63E−05-2.70E−02 | 36 |
| Embryonic Development | 4.24E−04-2.70E−02 | 24 |
| Hepatic System Development | 7.25E−04-2.70E−02 | 3 |
| and Function | | |

Top Canonical Pathways

| Name | p-value | Rat [symbol] o |
|---|---|---|
| 14-3-3-mediated Signaling | 1.4E−03 | 9/1 [symbol] (0.08) |
| Integrin Signaling | 3.38E−03 | 12/ [symbol] (0.061) |
| CXCR4 Signaling | 4.63E−03 | 10/ [symbol] (0.061) |
| Neuregulin Signaling | 6.58E−03 | 7/9 [symbol] (0.071) |
| VEGF Signaling | 7.02E−03 | 7/9 [symbol] (0.074) |

[symbol]

indicates data missing or illegible when filed

TABLE 8
Top age-biomarker gene networks and associated functions

| | | | Focus. | |
|---|---|---|---|---|
| Network | Molecules in Network | Score | Molecule | Top Functions |
| 1 | ↑ADAM17, ADCY, Calcineurin protein(s), ↓CALM3, Calmodulin, CaMK [symbol] , ↑CD44, | 39 | 25 | Cell-To-Cell Signaling and |
| | Ck2, Clathrin protein, ↓CRYM, ↓CX3CL1, ↑DLG1, ↓OLG3, ↓DUSP14, ↑GAB2, | | | Interaction, Cell-mediated |
| | ↑KCNJ2, ↓KCNQ3, ↑KIF13B, ↓MAPK1, Metalloprocease, | | | Immune Response, |
| | ↑MYO6, NMDA Receptor, ↓NRGN, ↑PICALM, ↑PIP4K2A, Po2b, ↓PPEF1, | | | Hematological System |
| | ↓PPP3CB, Ptk, ↓RIT2, ↑SEMA4C, ↑TJAP1, ↑VCAN, ↑ZHX2, ↑ZHX3 | | | Development an Function |
| 2 | ↑ADAMTS1, Ap1, Caspase ↑CDS9, ↑CDKN1C, ←CLDND1, ↑CLIC4, ↑CLU, | 37 | 24 | Nervous System |
| | ↓CRH, ↓CRIP2, Cyclin A, Cyclin E, Cytochrome c, ↓FAM162A, ↑H3F3A (includes | | | Development and Function, |
| | EG: 30201, hCC, Histone h3, insulin, ↑LDB3, ↑LPIN1, ↑MAL, ↓MBD1, ↑MECP2, | | | Tissue Development, |
| | Mck, ↑MLL, ↑MT1G, ↑MT2A, P3 [symbol] MAPK, ↑PIGA, ↓PRKCB, ↑RPS6KA5, | | | DNA Replication, |
| | Rsk, ↑SAFB2, ↑UNG, ↑VAMP3 | | | Recombination, and Repal |
| 3 | ↓ADRA2A, Alcohol group acceptor phosphotransferase, ↓CALB1, CD3, ↑CFLAR, ENaC, | 35 | 25 | Genetic Disorder, |
| | ↑HBP1, HDL, ↑HIPK2, IKK, ↑ITPKB, ↑LITAF, ↓MAP2K4, ↑MAP4K4, ↓NEK2, | | | Neurological Disease, |
| | Ntal, NfkB, ↑RARRES3, ↓RASGRF1, ↑RHOG, ↑SDC4, ↑SEMA3B, ↑SEPT4, | | | Cell Death |
| | ↓SERPINF1, ↑SGK1, ↑SLC1IA2, ↑ST18, TCR, ↓TOLLIP, ↓UBE2N, Ubiquitin, | | | |
| | VitaminD3-VDR-RXR, ↑WNK1, ↑WSB1, ↓WTAP | | | |
| 4 | ↓ACTN2, ↑AHCYL1, ALP, Alpha Actinin, Calpain, ↑CAPN3, ↑CSRP1, ↑DDR1, | 35 | 23 | Genetic Disorder, |
| | ↓DDK5, ERK, Fgr, ↑FGF1, ↓FGF13, C alpha1, ↓HOMER1, ↑LPAR1, ↑LTBP1, Mmp, | | | Neurological Disease, |
| | ↓NELL1, ↓NPPA, ↑PALLD, ↑PKD2 (includes EG: 5311), Pkg, PLC, PLC gamma, | | | Psychologica lDisorders |
| | ↓PLCB1, ↓PNOC, ↓PTPRR, ↑RASGRP3, ↓RGS4, Tgt beta, ↑TGFB3, ↑TNS1, | | | |
| | ↑TOB1, Tubulin | | | |
| 5 | 14-3-3, ↑AKAP1, ↓ATXN10, ↑BTG1, ↑C19ORF2, ↑C22ORF9, ↓DNAJA1, | 35 | 23 | Post-Translational |
| | ↓DR1, Dynamin, ↑EZH1, C alpha, G protein beta gamma, ↑GFAP, Gpcr, Hsp70, Hsp90, | | | Modification, Protein |
| | ↓HSPA8, IFN BetaΔ, Ink, ↑KIF5B, ↑NDRG1, ↓NRG1, ↑NUMA1, Proteasome, | | | Folding, Nervous System |
| | ↓RAP1GDS1, RNA polymerase II, ↓SNCA, STAT, ↑TCEB3, ↑TNPO1, | | | Development and Function |
| | ↑TOB2, ↑TSR1, ↓VDAC1, ↓YWHAZ, ↑ZNF451 | | | |

Genes in with an upward arrow (increased) or downward arrow (decreased) identify age biomarkers included in the networks. Other genes and molecular functions represent added indirect nodes in networks. Scores are negative log (p-value). The top functions indicate the biological functions and/or diseases that were most significant to the genes in the network. [symbol]
indicates data missing or illegible when filed

Additionally, nervous system development was a top category (70 associated genes, Table 7). Notably, this identified functional group included epigenetic regulators, transcription factors, and histones, consistent with a potential roles in regulating some of the observed transcript changes, and suggested the presence of a putative age-related transcriptional program as shown in Table 9 below.

TABLE 9
Epigenetic Regulators and Transcription Factors

| Gene | N | G | B | E (%) | P-val |
|---|---|---|---|---|---|
| Histone cluster 1, H2bk | | | 3 | −34 | 1E−16 |
| Down-regulator of Transcription 1 | | | 3 | −40 | 5E−11 |
| Methyl CpG Binding Protein 2 | | | 3 | +24 | 2E−10 |
| Mitochondrial ribosomal protein S12 | 3 | | | −39 | 2E−10 |
| Methyl-CpG Binding Domain 1 | 3 | | | −18 | 1E−06 |
| Chromatin Modifying Protein 1B | 3 | | | −18 | 4E−06 |
| Forkhead Box O1 | | 3 | | +42 | 2E−06 |
| H3 Histone, family 3A/3B | | 3 | | +73 | 2E−16 |
| Transcription Factor EB | | 3 | | +66 | 6E−09 |
| CREB-regulated Transcription Coactivator | | 3 | | +39 | 3E−10 |
| Jumonji, AT-rich Interactive Domain 1A | | 3 | | +42 | 1E−08 |
| HMG-box Transcription Factor 1 | | 3 | | +42 | 1E−11 |

Importantly, neurological disease was a top category (115 associated genes), supporting the hypothesis of disease promotion by normal aging (Table 7 and 8).

The age-related biosignature was further characterized using the microarray drug-matching program, C-MAP (Lamb, et al., Science, 313:1929-35 (2006)), by identifying drugs causing transcriptional changes in cell culture inversely correlating with the biosignature (candidate anti-aging drugs). As an internal validation, C-MAP identified known anti-aging and neuroprotective agents, such as α-estradiol and GW-8510, an inhibitor of neuronal apoptosis; shown in Table 10, shown below.

TABLE 10
C-MAP Candidate Brain Aging Drugs

| Drug | Mechanism | P-val |
|---|---|---|
| Anti-aging | | |
| GW-8510 | CDK2 inhibitor: neuroprotective | 1E−04 |
| α-estradiol | Estrogen enantiomer, neuroprotective | 1E−04 |
| Urapidil | Antihypertensive, α1 adrenergic antagonist, | 2E−04 |
| | α2 adrenergic agonist, 5HT1A agonist: | |
| | neroprotective | |
| Alsterpaullone | Inhibitor of CDK2, CDK1/Cyclin B, | 5E−04 |
| | CDK5/p25, GSK-3β, Tau phosphorylation: | |
| | neuroprotective | |
| Skimmianine | Furoquinoline alkaloid (plant extract) used in | 6E−04 |
| | folk medicine; potentially anti-inflammatory, | |
| | anti-tumorigenic, MAO inhibitor | |
| H-7 | PKC inhibitor, decreases calcium current, | 7E−04 |
| | alters astrocyte morphology | |
| Niacin | Anti-inflammatory properties: used to treat | 0.005 |
| (vitamin B3) | hypercholesterolemia, arteriosclerosis, and | |
| | cardiovascular disease | |
| Biotin | Regulates insulin secretion; therapeutic | 0.009 |
| (vitamin B7) | efficacy in diabetes | |
| Pro-aging | | |
| Wortmannin | Inhibitor of PI3-K cell survival pathway | 1E−06 |
| Benzamil | Na/Ca exchange blocker, inhibitor of NGF | 5E−04 |
| | mediated neurite outgrowth | |

Interestingly, results pointed to regulatory roles for cell cycle proteins and neurotransmitters as candidate anti-aging drugs, as two of the top six drugs were cyclin-dependent-kinase inhibitors and two were monoaminergic modulators.

### Example 3

### Neurological-Disease Related Genes are Overrepresented Amongst Age-Regulated Genes and Change in Pro-Disease Directions

Materials and Methods

Top 20 Ingenuity® Functional Categories Associated with Age-Regulated Genes (Figure Adapted from Ingenuity®).

Criteria for selection for age regulated genes were age-regression p<0.001 in at least one area or p<0.01 in two brain areas (n=3,935). The top four functions were largely driven by the six neurologic diseases focused on in the paper (AD, PD, ALS, HD, SCHZ and BPD). The top diseases in the top “genetic disease” category were all age-related and included autoimmune disease (529 genes), coronary artery disease (291 genes), bipolar disorder (285 genes), insulin-dependent diabetes mellitus (270 genes), Huntington's disease (267 genes), Alzheimer's disease (187 genes), Parkinson's disease (170 genes), amyotrophic lateral sclerosis (170 genes), schizophrenia (161 genes), prostate cancer (113 genes), colon cancer (103 genes), and autism (27 genes). Top Neurologic diseases were the six aforementioned and also included several types of brain cancer, autism, and epilepsy. The third ranked category, Skeletal and muscular disorders, were largely driven by PD and HD, which ingenuity considers to be in this category. The 421 genes associated with psychological disorders were almost entirely driven by Schizophrenia and Bipolar disorder.

Age-Regulated Genes Associated with the Top Six Neurological Diseases.

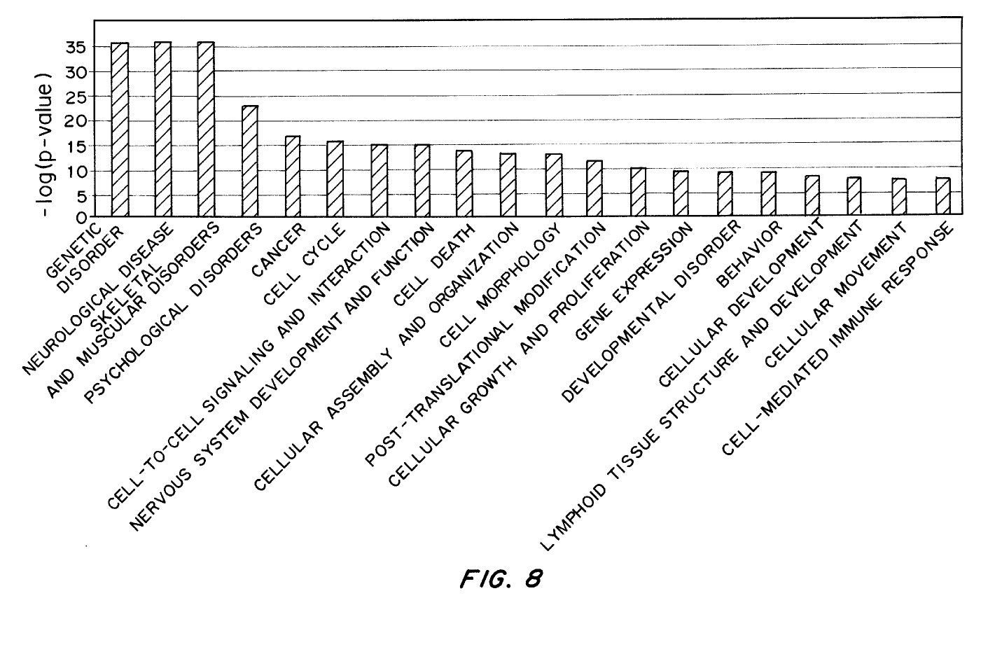

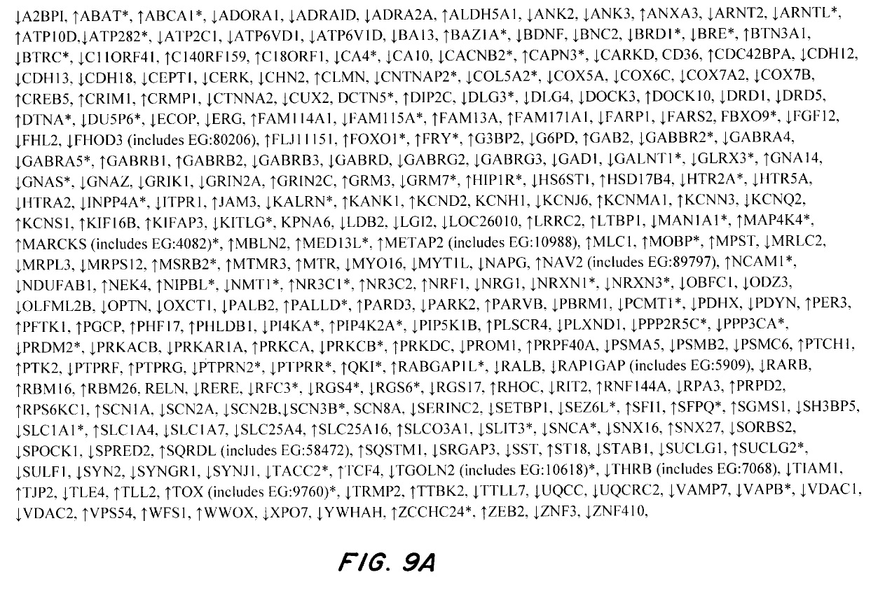

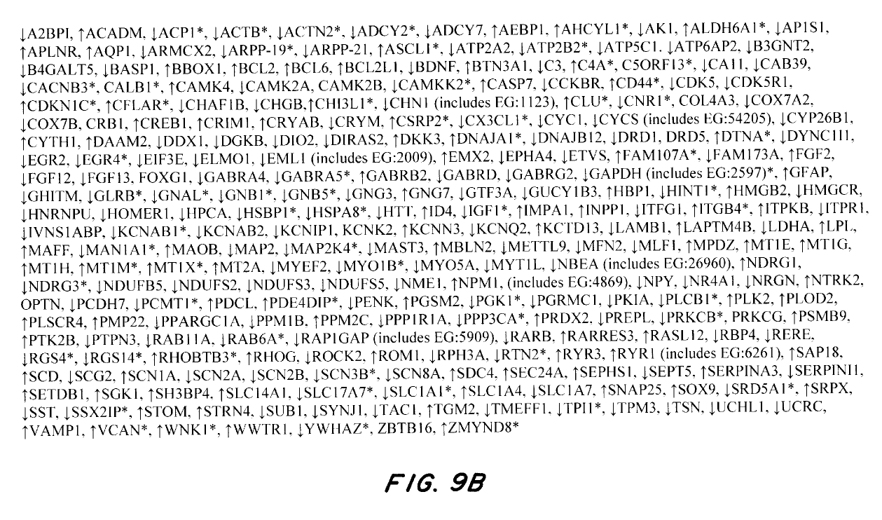

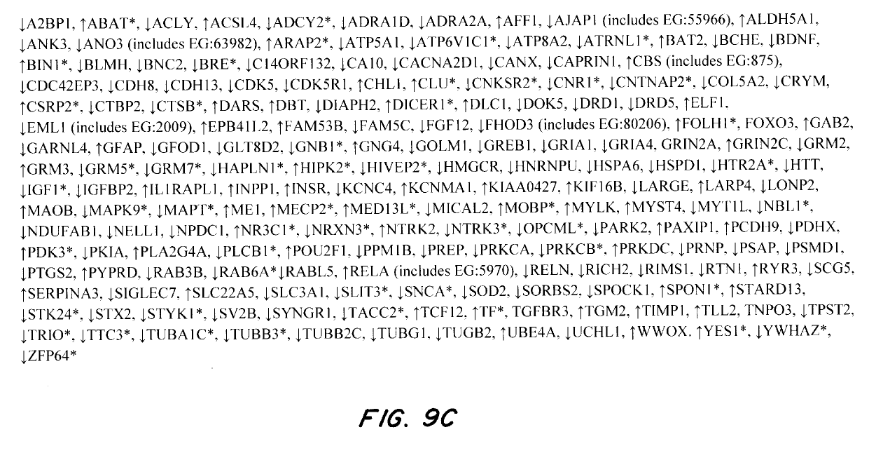

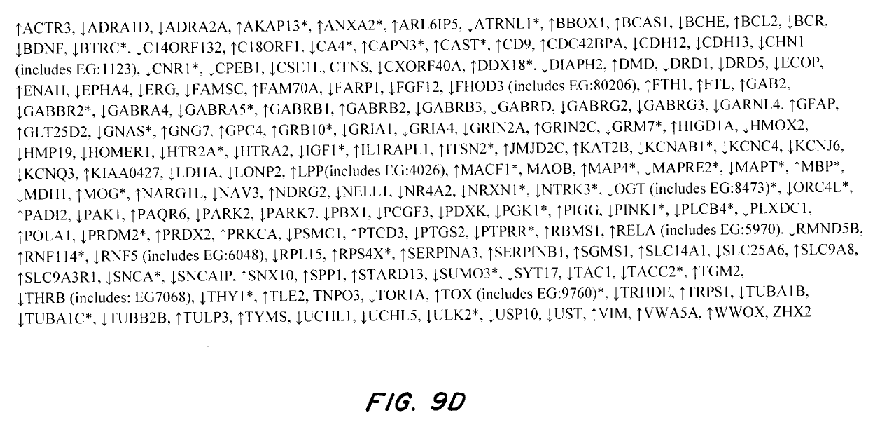

Disease associations are based on Ingenuity's database of hand-curated literature searches performed by PhD level scientists. Depicted direction of age-regulated changes are from the ACC dataset and are not necessarily congruent with the direction of change in the other three brain areas (FIGS. 9A-F). Genes with asterisks have more than one probeset per gene represented in the selection. Directions of change for these genes are that of the probeset with the most significant p-value.

Top 20 Ingenuity® Functional Categories Analysis of Genes that were not Age-Regulated.

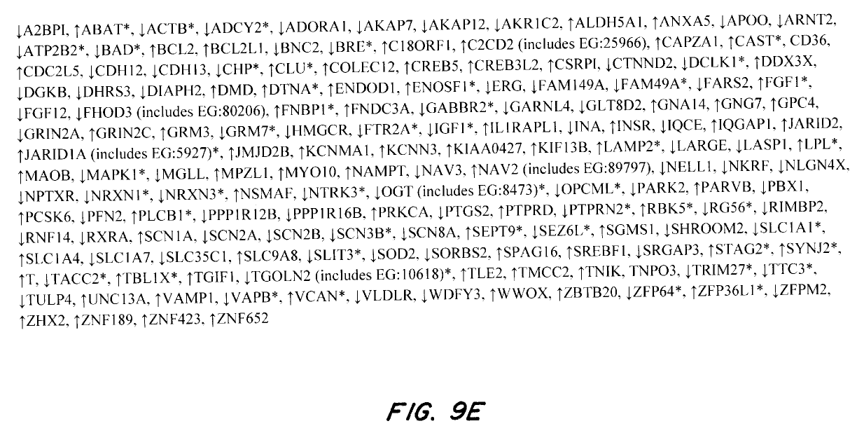

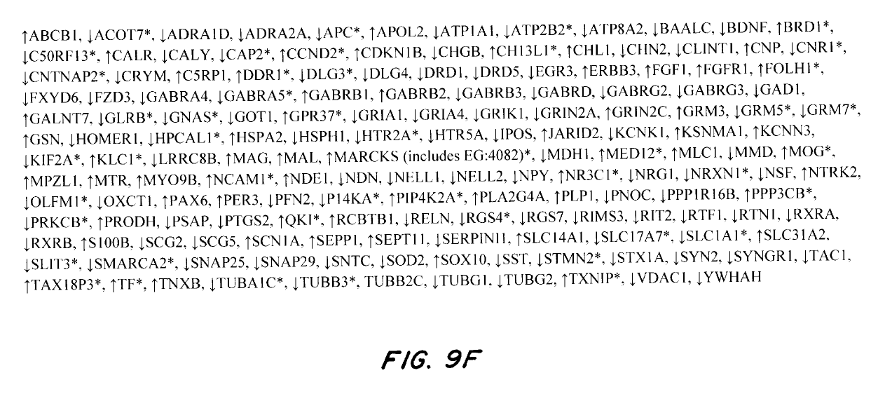

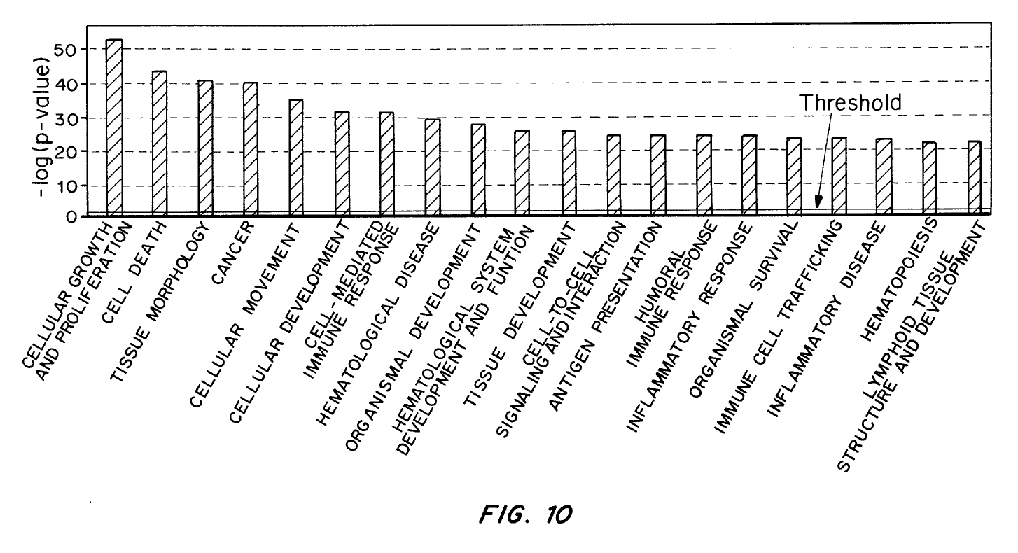

Criteria for non-age-regulated genes were p>0.05 in all four brain areas (n=7790) (FIG. 10). The top category of non-age regulated genes was cellular growth and proliferation, which is logical for the non-dividing tissue of the brain. Notably, neurologic disease was not in the top 20 categories, ranking 44^{th}.

Results

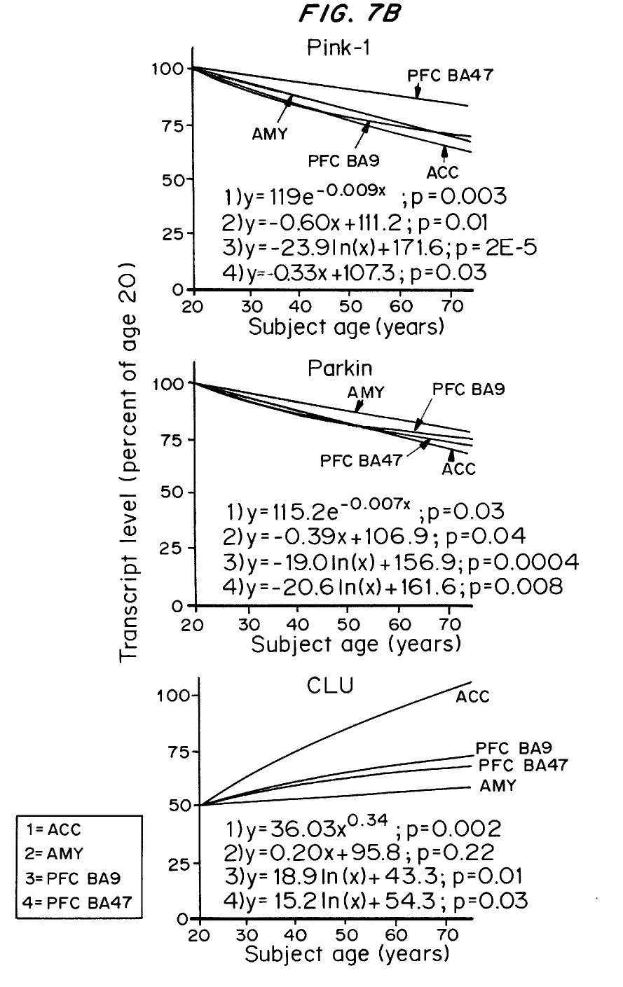

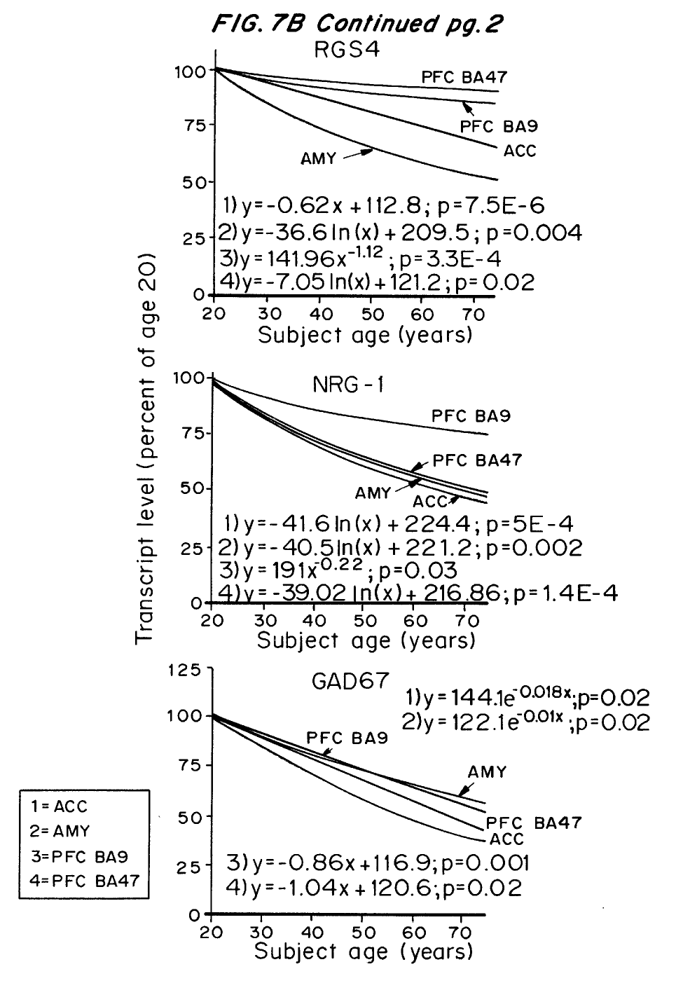

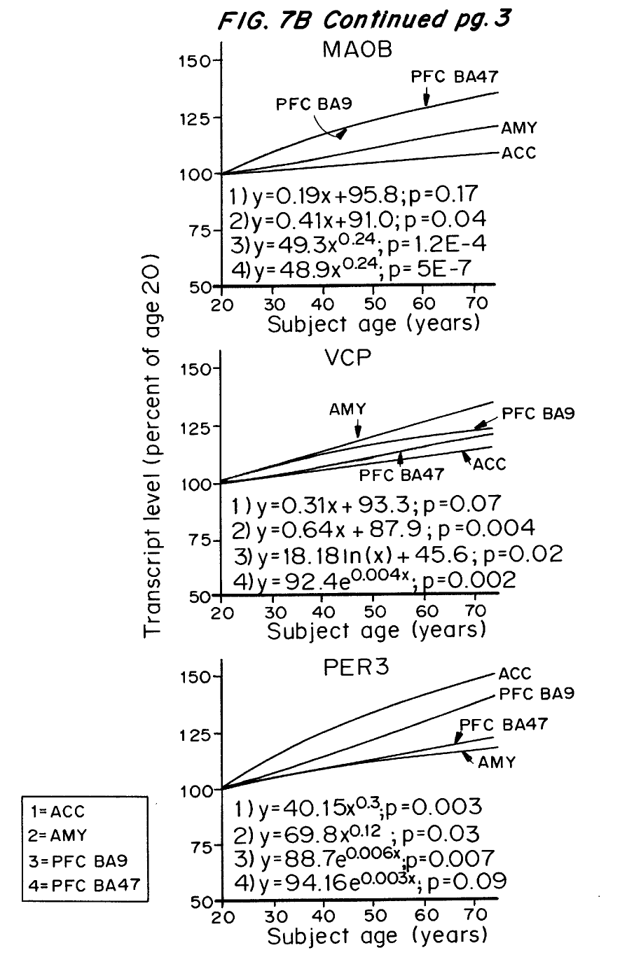

To characterize the extent of overlap between age and disease pathways, a wider gene group (n=3,935) not restricted by significance in all brain areas (p<0.001 in one area, or p<0.01 in two) was selected. Again, neurological disease was a top Ingenuity®-identified functional category, comprising 34% of age-regulated genes (FIG. 7A, FIG. 8). The top indentified diseases, Alzheimer's, Parkinson's, Huntington's, amyotrophic lateral sclerosis, schizophrenia and bipolar disorder were all common neurological diseases with well-defined ages of onset (FIGS. 9A-F). Conversely, disease-associated genes represented only 4% of non-age-regulated genes (p>0.05 in all areas), and neurological disease fell to the 44^{th} functional category with no specific diseases represented (FIG. 7A, FIG. 10). Furthermore, investigations into a subset of genes with well-established disease-associations revealed that expression changes were almost unanimously (32/33) in disease-promoting directions (FIG. 7B, Tables 11 and 12, shown below).

TABLE 11
Disease gene age-regulation.

| | Direction of | | | | | | | | | |
|---|---|---|---|---|---|---|---|---|---|---|
| | change in disease | | | | | | Change with age (%) (p-val) | | | |
| Disease-associated gene_symbol | AD | PD | HD | ALS | SCZ | BPD | ACC | AMY | PFC BA9 | PFC |
| Amyloid beta precursor | u | | | | | | −17.4(0.005) | n.s. | −25.9(1.2E−5) | n.s |
| protein binding-1_Fe65 | | | | | | | | | | |
| Amyloid beta precursor | ↑ | | | | | | +18.1(0.02) | +10.1(0.04) | +22.1(0.0003) | n.s. |
| protein binding-2_APPB2/PAT1 | | | | | | | | | | |
| Amyloid precursor-like protein | ↓ | | | | | | −25.4(0.009) | n.s. | −31.1(0.0001) | −30.5(0.008) |
| 2_APLP2 | | | | | | | | | | |
| Clusterin/Apolipoprotein-J_CLU | ↑ | | ↑ | ↑ | | | +80.5(0.0004) | +29.3(0.02) | +75.8(1.3E−7) | +54.8(5.8E−8) |
| Monoamine Oxidase B_MAOB | ↑ | ↑ | ↑ | ↑ | | | n.s. | +20.5(0.42) | +34.2(0.00006) | +34.9(8.5E−7) |
| Microtubule-associated protein | ↑ | ↑ | | | | | −34.9(0.009) | n.s. | −28.7(2.9E−6) | n.s. |
| tau_MAPT | | | | | | | | | | |
| □-synuclein_□-syn | ↓ | u | | | | | −32.6(8.8E−5) | −39.4(0.03) | −19.7(1.7E−6) | −20.3(0.001) |
| Parkinson Disease-2_Parkin | u | ↓ | | | | | −29.5(0.02) | −19.4(0.04) | −23.9(0.0003) | −26.0(0.009) |
| Parkinson Disease-5_UCHL1 | | ↓ | | | | | −27.2(0.001) | −24.6(0.02) | −14.9(0.002) | n.s. |
| Parkinson Disease-6_PINK-1 | | ↓ | | | | | −36.2(0.003) | −29.9(0.009) | −29.8(6.3E−6) | −15.7(0.03) |
| Parkinson Disease-7_DJ-1 | | ↓ | | | | | −25.9(0.0006) | −15.3(0.02) | n.s. | n.s. |
| Parkinson Disease-13_HTRA2 | | ↓ | | | | | −27.4(0.002) | n.s. | −9.5(0.04) | −25.6(0.0006) |
| Huntingtin_HD | | | ↓ | | | | n.s. | n.s. | −22.9(0.0005) | n.s. |
| Valosin-containing protein_VCP | | | ↑ | | | | n.s. | +32.5(0.003) | +22.9(0.001) | +22.9(0.002) |
| Mitochondrial Complex 1 | | | ↓ | | | | −22.0(0.001) | −28.9(0.009) | n.s. | n.s. |
| Subunit_NDUFB5 | | | | | | | | | | |
| Mitochondrial Complex 1 | | | ↓ | | | | −33.0(0.0003) | n.s. | −16.8(0.002) | n.s. |
| Subunit_NDUSF2 | | | | | | | | | | |
| Mitochondrial Complex 1 | | | ↓ | | | | −24.4(6.4E−5) | −22.1(0.05) | −15.4(0.01) | −13.9(0.005) |
| Subunit_NDUSF3 | | | | | | | | | | |
| Mitochondrial Complex 1 | | | ↓ | | | | −17.9(0.0007 | n.s. | n.s. | n.s. |
| Subunit_NDUSF3 | | | | | | | | | | |
| Mitochondrial Complex 4 | | | ↓ | | | | −22.7(0.0004) | −27.2(0.004) | n.s. | n.s. |
| Subunit_COX7B | | | | | | | | | | |
| Cyclin-dependent Kinase-5_CDK5 | ↓ | | ↓ | | | | −35.3(4E−5) | n.s. | −30.9(1.9E−8) | −25.6(0.0002) |
| NF-kappa B_NF-kB | ↑ | ↑ | ↑ | ↑ | | ↑ | +16.2(0.03) | n.s. | +24.0(0.0001) | +15.1(0.01) |
| Manganese Superoxide | ↓ | | | ↓ | ↓ | | n.s. | n.s. | −50.3(0.0007) | n.s. |
| dismutase_SOD2 | | | | | | | | | | |
| Cholecystokinin_CCK | ↓ | ↓ | | | ↓ | | −33.8(0.002) | −29.7(0.03) | −18.1(0.01) | n.s. |
| Neuropeptide-Y_NPY | ↓ | u | | | ↓ | ↓ | −33.8(0.02) | −41.7(0.008) | −34.1(0.003) | n.s. |
| Cannabanoid Receptor-1_CB1 | ↓ | u | ↓ | | ↓ | | n.s. | n.s. | −45.7(2.6E−10) | −39.4(0.002) |
| Parvalbumin_PVALB | ↓ | u | | | ↓ | ↓ | −58.6(0.001) | n.s. | n.s. | −34.5(0.02) |
| Glutamate decarboxybse I_GAD67 | | ↓ | | | ↓ | ↓ | −59.3(0.02) | −39.3(0.02) | −43.2(0.0009) | −51.9(0.02) |
| GABA transaminase_GABA-T | u | | | | ↑ | | +25.3(0.04) | n.s. | +28.4(0.0003) | n.s. |
| Brain-derived neurotrophic | ↓ | ↓ | ↓ | | ↓ | ↓ | −45.1(0.0005) | n.s. | −39.8(8.9E−6) | −41.8(3.4E−5) |
| factor_BDNF | | | | | | | | | | |
| Serotonin 2A Receptor_HTR2A | ↓ | | | | ↓ | ↓ | −40.8(0.0007) | +64.9(0.04) | −39.4(0.0001) | −46.3(0.0008) |
| Serotonin 5A Receptor_HTR5A | | | | | u | u | −39.3(0.0007) | −32.9(0.00005) | −33.2(0.0001) | −34.3(0.05) |
| Somatostatin_SST | ↓ | ↓ | ↓ | | ↓ | | −45.0(0.0001) | −61.4(0.01) | −57.3(5.4E−6) | −39.4(0.001) |
| Regulator of G-protein signaling- | ↓ | | ↓ | | ↓ | u | −43.5(0.008) | −75.3(0.006) | −44.7(2.0E−8) | −57.5(2.1E−5) |
| 4_RGS4 | | | | | | | | | | |
| Reelin_RELN | u | u | | | ↓ | ↓ | −33.0(0.02) | n.s. | n.s. | −38.1(0.0002) |
| Neuregulin-1_NRG1 | u | | | | u | u | −52.3(0.0003) | −50.7(0.001) | −23.7(0.03) | −48.9(0.0002) |
| Dopamine Receptor DI_DRD1 | u | | ↓ | | u | u | −50.3(0.008) | n.s. | −33.7(0.001) | −48.7(0.006) |
| GABA receptor. alpha-5 | | | ↓ | | u | u | −48.3(0.03) | −59.4(0.02) | −67.0(8.3E−10) | −58.9(0.0003) |
| subunit_GABRA5 | | | | | | | | | | |
| Period homolog-3_PER3 | | | | | u | u | +46.7(0.002) | n.s. | +35.0(0.004) | n.s. |
| Aryl hydrocarbon receptor nuclear | | | | | u | u | −37.0(0.005) | n.s. | −44.5(1.4E−5) | −59.3(1.4E−5) |
| translocator-like_BMAL1 | | | | | | | | | | |

Agreement of directions between disease-related and age-regulated (age 70-age 20) gene expression changes.
↓ = decreased mRNA/protein levels reportedly pro-disease;
↑ = increased mRNA/protein levels reportedly pro-disease;
u = unknown/unclear reports of directionality in disease (references in supplementary Table 12);
n.s. = non-significant (p > 0.05) change with age.

TABLE 12
References for Direction of Neurologic Disease Expression Changes
in Disease (Supporting Table 9).

| | Direction of Change | | | | | | |
|---|---|---|---|---|---|---|---|
| | in Disease | | | | | | |
| Disease-associated gene_symbol | AD | PD | HD | ALS | SCZ | BPD | References for Pro-disease Directions |
| Amyloid beta precursor protein binding-1_Fe65 | u | | | | | | FE65 mRNA levels in AD human brain are decreased in |
| | | | | | | | cortex, however this appears to be cell type and brain |
| | | | | | | | region dependent; additionally it is unclear whether loss |
| Amyloid beta precursor protein | ↑ | | | | | | Involved in APP transport/processing; overexpression |
| binding-2_APPB2/PAT1 | | | | | | | results in Aβ accumulation [24, 25] |
| Amyloid precursor-like protein 2_APLP2 | ↓ | | | | | | Decreased mRNA levels in AD brain [26, 27] |
| Clusterin/Apolipoprotein-J_CLU | ↑ | | ↑ | ↑ | | | Increased mRNA levels in AD brains, HD striatum, and |
| | | | | | | | ALS spinal cord [28, 29, 30, 31] |
| Monoamine Oxidase B_MAOB | ↑ | ↑ | ↑ | ↑ | | | Increased mRNA levels in AD Cortex, HD Caudate and |
| | | | | | | | ALS spinal cord, also increased activity in PD brain. |
| Microtubule-associated protein tau_MAPT | ↑ | ↑ | | | | | MAO-B inhibitors are a common treatment for PD |
| | | | | | | | AD and PD associated with higher mRNA levels, |
| | | | | | | | polymorphic haplotypes and toxic gain of function |
| | | | | | | | mutations [36, 37] |
| □-synuclein_ □-syn | ↓ | u | | | | | Decreased mRNA levels in AD PFC; |
| | | | | | | | increased/decreased in PD brain [38, 39] |
| Parkinson Disease-2_Parkin | ↓ | ↓ | | | | | Associated with low expressing promoter |
| | | | | | | | polymorphisms in PD, and decreased mRNA levels in |
| | | | | | | | AD brain; also Parkin levels prevents Aβ accumulation |
| Parkinson Disease-5_UCHL1 | ↓ | ↓ | | | | | Decreased mRNA levels in PD and AD brains [41] |
| Parkinson Disease-6_Pink1 | | ↓ | | | | | Loss of function mutations cause familial PD; knock- |
| | | | | | | | down of Pink-1 in cell lines causes PD-like |
| Parkinson Disease-7_DJ-1 | | ↓ | | | | | Loss of function mutations in familial PD, decreased |
| | | | | | | | mRNA levels in PD Substantia Nigra[45] |
| Parkinson Disease-13_HTRA2 | | ↓ | | | | | Loss of function mutations in PD, loss of function |
| | | | | | | | mutations cause mitochondrial dysfunction and |
| | | | | | | | neurodegeneration in mice [46, 47] |
| Huntingtin_HD | | | ↓ | | | | Loss of WT Huntington is pro-disease as Huntingtin KO |
| | | | | | | | mice have a neurodegenerative phenotype, loss of wt |
| | | | | | | | huntingtin causes more severe/rapid degeneration and |
| | | | | | | | death in HD YAC128 mouse model, and the addition of |
| | | | | | | | wt huntingtin to mutant HD cell lines reduces cellular |
| Valosin-containing protein_VCP | | | ↑ | | | | Mutant VCP is associated with Paget's disease. While |
| | | | | | | | the levels of increased or decreased wt VCP is unknown |
| | | | | | | | in disease, a drosophila overepression model suggests |
| | | | | | | | that increased VCP would increase aggregate formation |
| Mitochondrial Complex 1 Subunit_NDUFB5 | | | ↓ | | | | Decreased mRNA levels in HD caudate [33] |
| Mitochondrial Complex 1 Subunit_NDUSF2 | | | ↓ | | | | Decreased mRNA levels in HD caudate [33] |
| Mitochondrial Complex 1 Subunit_NDUSF3 | | | ↓ | | | | Decreased mRNA levels in HD caudate [33] |
| Mitochondrial Complex 1 Subunit_NDUSF3 | | | ↓ | | | | Decreased mRNA levels in HD caudate [33] |
| Mitochondrial Complex 4 Subunit_COX7B | | | ↓ | | | | Decreased mRNA levels in HD caudate [33] |
| Cyclin-dependent Kinase-5_CDK5 | ↓ | | ↓ | | | | HD is Associated with decreased CDK5 protein in |
| | | | | | | | striatum and AD hippocampus [52, 53] |
| NF-kappa B_NF-kB | ↑ | ↑ | ↑ | ↑ | | ↑ | Increased mRNA levels in ALS Spinal Cord, AD |
| | | | | | | | hippocampus, PD brainstem and midbrain, BPD cortex, |
| | | | | | | | cultured HD neurons, HD mouse model [54, 55, 56, 57, 58] |
| Manganese Superoxide dismutase_SOD2 | ↓ | | | ↓ | | | Associated polymorphisms with AD; knock-down |
| | | | | | | | accelerates disease progression in AD and ALS mouse |
| Cholecystokinin_CCK | ↓ | ↓ | | | ↓ | | Decreased mRNA levels in SCZ, AD and PD PFC |
| Neuropeptide-Y_NPY | ↓ | u | | | ↓ | ↓ | Decreased mRNA levels in SCZ, BPD, and AD cortex |
| | | | | | | | [61, 63, 64, 65] |
| Cannabanoid Receptor-1_CB1 | ↓ | u | ↓ | | ↓ | | Decreased mRNA and protein levels in SCZ PFC, |
| | | | | | | | decreased mRNA in HD Globus Pallidus and AD |
| | | | | | | | caudate, increased/decreased in PD brain [66, 67, 68, 69] |
| Parvalbumin_PVALB | ↓ | u | | | ↓ | ↓ | Decreased mRNA levels in SCZ PFC, AD |
| | | | | | | | parahippocampal gyrus, and BPD cortex. In PD there is |
| | | | | | | | reports of decreased levels in globus pallidus and |
| | | | | | | | substantia nigra and PV is decreased in a Parkinsonian |
| | | | | | | | mouse model. However, there is one report of increased |
| | | | | | | | PV mRNA levels in PD Substantia Nigra |
| Glutamate decarboxylase 1_GAD67 | | ↓ | | | ↓ | ↓ | Decreased mRNA levels in PD globus pallidus and SCZ |
| | | | | | | | PFC [61, 77] |
| GABA transaminase_GABA-T | u | | | | ↑ | | Increased mRNA levels in SCZ cortex and |
| | | | | | | | increased/decreased AD brain [61, 78, 79, 80] |
| Brain-derived neurotrophic factor_BDNF | ↓ | ↓ | ↓ | | ↓ | ↓ | Decreased mRNA levels in SCZ PFC, BPD hippocampus |
| | | | | | | | and decreased in mRNA & protein in multiple brain |
| | | | | | | | areas in AD, PD, and HD [81, 82, 83, 84] |
| Serotonin 2A Receptor_HTR2A | ↓ | | | | ↓ | ↓ | Decreased mRNA levels in SCZ, BPD, and AD Cortex |
| | | | | | | | [symbol] |
| Serotonin 5A Receptor_HTR5A | | | | | u | u | Associated polymorphisms in SCZ and BPD-direction |
| | | | | | | | of mRNA levels changes have not been investigated |
| Somatostatin_SST | ↓ | ↓ | ↓ | | ↓ | | Decreased mRNA levels in SCZ, AD and PD Cortex and |
| | | | | | | | HD striatum [61, 63, 81, 91, 92] |
| Regulator of G-protein signaling-4_RGS4 | ↓ | | ↓ | | ↓ | u | Decreased mRNA levels in SCZ and AD PFC, multiple |
| | | | | | | | PD brain areas, and HD Striatum [93, 94, 95, 96] |
| Reelin_RELN | u | u | | | ↓ | ↓ | Decreased mRNA levels in SCZ, BPD, PD and AD |
| | | | | | | | cortex [97, 98, 99] |
| Neuregulin-1_NRG1 | u | | | | u | u | Reports are mixed as to whether NRG1 mRNA levels are |
| | | | | | | | increased or decreased in SCZ and are isoform specific; |
| | | | | | | | NRG1 polymorphisms are associated with BPD and |
| | | | | | | | psychosis in AD but direction of mRNA level changes |
| Dopamine Receptor DI_DRD1 | u | | ↓ | | u | u | Decreased mRNA levels in SCZ hippocampus; linkage |
| | | | | | | | to DRD1 haplotypes in BPD with direction of levels |
| | | | | | | | changes not investigated [105, 106] |
| GABA receptor, alpha-5 subunit_GABRA5 | | | ↓ | | u | u | Decreased mRNA levels in HD caudate; associated |
| | | | | | | | polymorphisms with BPD and age of onset in SCZ- |
| | | | | | | | direction of associated levels changes have not been |
| Period homolog-3_PER3 | | | | | u | u | Associated polymorphisms with BPD and SCZ-direction |
| | | | | | | | of mRNA level changes have not been investigated |
| | | | | | | | [109, 110] |
| Aryl hydrocarbon receptor nuclear translocator- | | | | | u | u | Associated polymorphisms in SCZ and BPD-direction of |
| like_BMAL1 | | | | | | | mRNA level changes have not been investigated |
| | | | | | | | [109, 111] |

[symbol]

indicates data missing or illegible when filed

Examples of age-regulated plots for specific disease-related gene are shown in FIG. 7B, which shows a discrepancy in rates observed across brain regions for some genes. For instance, clusterin (CLU), an Alzheimer-related gene displayed greatest age-related increase in ACC, where neuregulin (NGG-1), a schizophrenia-related gene, showed lowest age-related downregulation in BA9 (FIG. 7B), together providing a potential mechanisms for region-specific onset of pathological symptoms.

### Example 4

### SIRT5ₚᵣₒₘ₂ Associates with Decreased SIRT5 Expression and Accelerated Molecular Aging, Particularly of Mitochondrial-Localized Proteins, in a Brain Area-Specific Manner

The hypothesis that longevity genes may regulate brain aging and that polymorphisms in these genes may influence gene sets involved in risk for disease was tested. Five polymorphisms in three candidate longevity genes (FIG. 5, Table 3) were assessed initially, but the rest of the study was focused on a SIRT5ₚᵣₒₘ₂ single nucleotide polymorphism (snp), as it was associated with the largest and most statistically robust effects on molecular aging (Table 13, shown below).

TABLE 13
Significance of genotypic effects on molecular age in ACC and AMY

| | Intersection | | | Difference in |
|---|---|---|---|---|
| | transcripts: | Molecular | | total molecular |
| | n in | Years | | age using all |
| | pro-aging | Different | Intersection | age transcripts |
| | direction/total | (p-val) | FDR | (p-val) |
| ACC | | | | |
| Sirt 5 | 227/231 | CC +24 | 19% | CC +9.1 |
| prom2 | | (0.0001) | | yrs (0.004) |
| Klotho | 7/9 | VS +11.1 | 100% | VS +0.46 (0.9) |
| KL-VS | | (0.12) | | |
| HTR1B | 18/23 | GG +9.8 (0.12) | 100% | GG +7.9 (0.09) |
| AMY | | | | |
| Sirt 5 | 30/48 | CT +2.0 (0.55) | 100% | CT +2.2 (0.69) |
| prom2 | | | | |
| Klotho | 38/39 | VS +23.6 | 100% | VS +4.7 (0.18) |
| KL-VS | | (0.001) | | |
| HTR1B | 14/24 | GG +5.3 (0.25) | 100% | GG −0.6 (0.86) |

SIRT5 was selected due to the increasing role of the sirtuin gene family in neurodegenerative disease (Gan, et al., Neuron, 58:10-4 (2008)) and due to the previous observation of altered Sirt5 expression in htr1b^{KO} mouse cortex, a mouse model with anticipated brain aging (Sibille, et al., Mol Psychiatry, 12:1042-56 (2007)). The SIRT5ₚᵣₒₘ₂ was identified as a snp of interest, due to its location in a mouse/human conserved region predicted by two separate programs to contain a promoter region (FIG. 5). The studies were concentrated on cohort 2 subjects, for which genetic material was available.

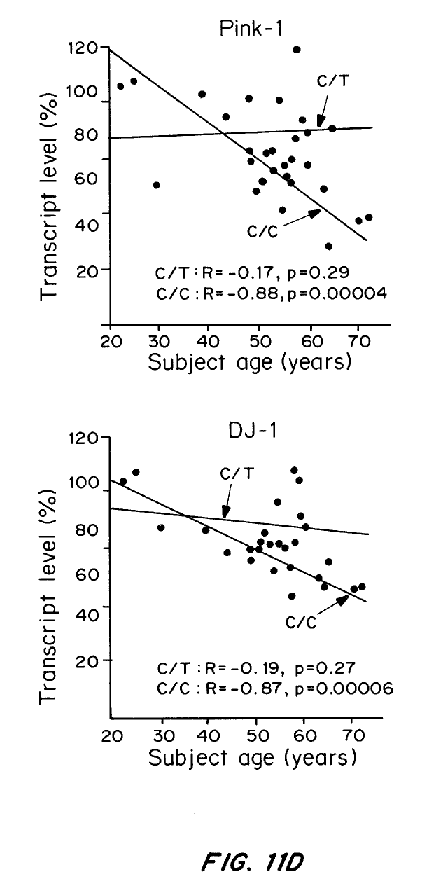

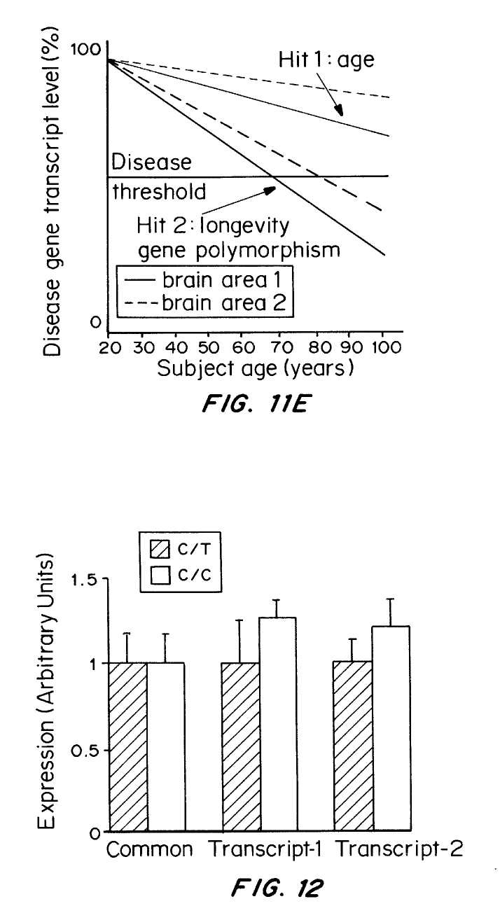

The data (qPCR) shows that the SIRT5_{Prom2} polymorphism associates with a 45-55% decrease in expression in two SIRT5 transcript variants in ACC (FIG. 11A). SIRT5 itself did not display age-regulated expression levels (age-regression p=0.45), thus genotypic differences in expression were present at all ages. No SIRT5_{Prom2} genotype effect on SIRT5 expression was observed in amygdala (FIG. 12), suggesting a brain-region specific effect of SIRT5_{Prom2}. SIRT5 C/C (low-expresser) allele carrier subjects had significantly older ACC molecular ages (+8.6 years, p=0.003, FIG. 11B) compared to C/T carriers. The observed difference was not due to residual age effect, as the C/C and C/T allele carrier cohorts were rigorously matched for chronological age. Instead, the difference resulted from apparent accelerated ACC molecular aging rates in C/C carriers (increased molecular vs. chronological age slope, FIG. 11B). Using an amygdala-specific biosignature, the data show that SIRT5ₚᵣₒₘ₂ was not associated with altered amygdala molecular aging, consistent with the fact that the SIRT5ₚᵣₒₘ₂ was not associated with altered SIRT5 level in that brain region (Table 13).

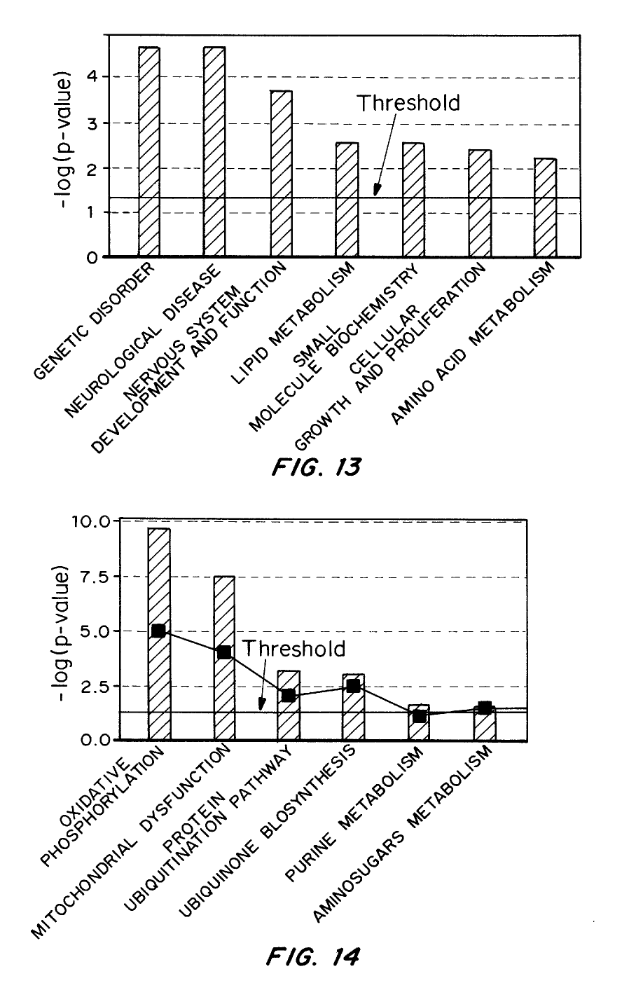

The question of whether SIRT5ₚᵣₒₘ₂'s correlation with older molecular ages was global or potentially driven by a subset of genes was investigated. The significance of SIRT5ₚᵣₒₘ₂ genotype association with transcript changes for all other genes in well age-matched subgroups was determined, as an exploratory approach for potential indirect SIRT5ₚᵣₒₘ₂-mediated effects (FIG. 13). SIRT5ₚᵣₒₘ₂ associated (p<0.01) with altered levels for 972 transcripts, including 231 age-regulated transcripts (FIG. 7B). These latter transcript changes almost unanimously (98%) associated with older molecular ages in SIRT5-C/C carriers. Indeed, based on these “core” SNP-by-age intersection transcripts, subjects carrying the C/C allele were on average 24.1 molecular years older than C/T carriers (p=0.0004, FIG. 11B). These core transcripts possibly represent proximal effectors in SIRT5's putative modulation of age-related expression changes.

These predominantly (74%) neuronally-enriched transcripts included potential brain-aging regulators, transcription factors (GTF3A, TCF7L2), Histone 3 (H3F3A/3B), Chromatin Modifying Protein 2A (CHMP2A), and CDK5 (Table 14, shown below).

TABLE 14
Mitochondrial Age-regulated Transcripts Affected By Sirt5
Genotype in ACC.

| Probeset | | Gene | CC-CT | CC-CT | Snp | Age, | Age | | | |
|---|---|---|---|---|---|---|---|---|---|---|
| ID | Gene Name | Symbol | (%) | (years) | P-val | 70-20(%) | P-val | N | G | B |
| 210149_s_at | ATP synthase, H+ transporting, | ATP5H | −17.2 | 31.6 | 6.2E−04 | −27.2 | 8.1E−03 | 1 | 0 | 0 |
| | mitochondrial F0 complex, subunit d | | | | | | | | | |
| 208678_at | ATPase, H+ transporting, lysosomal | ATP6V1E1 | −13.3 | 25.6 | 9.4E−03 | −25.9 | 5.6E−03 | 0 | 0 | 1 |
| | 31 kDa, V1 subunit E1 | | | | | | | | | |
| 203880_at | COX17 cytochrome c oxidase | COX17 | −14.9 | 28.7 | 6.7E−03 | −25.9 | 7.5E−03 | 0 | 0 | 1 |
| | assembly homolog (S. cerevisiae) | | | | | | | | | |
| 202698_x_at | cytochrome c oxidase subunit IV | COX4I1 | −11.9 | 23.4 | 2.3E−03 | −25.4 | 2.0E−03 | 0 | 0 | 1 |
| | isoform 1 | | | | | | | | | |
| 213735_s_at | cytochrome c oxidase subunit Vb | COX5B | −11.8 | 32.5 | 3.3E−03 | −18.1 | 8.4E−03 | 0 | 0 | 1 |
| 201441_at | cytochrome c oxidase subunit Vib | COX6B1 | −13.7 | 31.0 | 1.5E−03 | −22.1 | 5.1E−03 | 0 | 0 | 1 |
| | polypeptide 1 (ubiquitous) | | | | | | | | | |
| 202110_at | cytochrome c oxidase subunit VIIb | COX7B | −11.3 | 25.0 | 9.1E−04 | −22.7 | 3.8E−04 | 1 | 0 | 0 |
| 201066_at | cytochrome c-1 | CYC1 | −11.3 | 31.3 | 1.4E−03 | −18.1 | 2.2E−03 | 0 | 0 | 1 |
| 205012_s_at | hydroxyacylglutathione hydrolase | HAGH | −16.6 | 30.6 | 1.1E−03 | −27.2 | 3.1E−03 | 1 | 0 | 0 |
| 213132_s_at | malonyl CoA:ACP acyltransferase | MCAT | −23.5 | 32.4 | 4.2E−03 | −36.2 | 7.1E−03 | 1 | 0 | 0 |
| | (mitochondrial) | | | | | | | | | |
| 213333_at | malate dehydrogenase 2, NAD | MDH2 | −14.2 | 24.0 | 4.5E−03 | −29.5 | 1.5E−03 | 1 | 0 | 0 |
| | (mitochondrial) | | | | | | | | | |
| 204386_s_at | mitochondrial ribosomal protein 63 | MRP63 | −10.6 | 32.3 | 3.5E−03 | −16.4 | 6.3E−03 | 0 | 0 | 1 |
| 224330_s_at | mitochondrial ribosomal protein L27 | MRPL27 | −10.8 | 29.8 | 7.6E−03 | −18.1 | 7.1E−03 | 0 | 0 | 1 |
| 224331_s_at | mitochondrial ribosomal protein L36 | MRPL36 | −15.9 | 27.0 | 2.7E−05 | −29.5 | 6.1E−04 | 0 | 0 | 1 |
| 203152_at | mitochondrial ribosomal protein L40 | MRPL40 | −11.4 | 35.1 | 1.4E−03 | −16.2 | 6.4E−03 | 0 | 0 | 1 |
| 223480_s_at | mitochondrial ribosomal protein L47 | MRPL47 | −13.6 | 23.9 | 4.3E−04 | −28.4 | 1.6E−04 | 0 | 0 | 1 |
| 201717_at | mitochondrial ribosomal protein L49 | MRPL49 | −7.2 | 23.4 | 7.6E−03 | −15.4 | 3.9E−03 | 0 | 0 | 1 |
| 211595_s_at | mitochondrial ribosomal protein S11 | MRPS11 | −15.2 | 25.7 | 9.5E−03 | −29.5 | 6.8E−03 | 0 | 0 | 1 |
| 224948_at | mitochondrial ribosomal protein S24 | MRPS24 | −16.6 | 28.2 | 5.7E−03 | −29.5 | 8.6E−03 | 1 | 0 | 0 |
| 220688_s_at | mRNA turnover 4 homolog | MRTO4 | −12.2 | 20.7 | 3.1E−03 | −29.5 | 8.1E−04 | 1 | 0 | 0 |
| 218160_at | NADH dehydrogenase (ubiquinone) | NDUFA8 | −13.3 | 25.6 | 7.4E−03 | −25.9 | 1.7E−03 | 1 | 0 | 0 |
| | 1 alpha subcomplex, 8, 19 kDa | | | | | | | | | |
| 218200_s_at | NADH dehydrogenase (ubiquinone) | NDUFB2 | −12.5 | 21.1 | 8.8E−03 | −29.5 | 1.2E−03 | 0 | 0 | 1 |
| | 1 beta subcomplex, 2, 8 kDa | | | | | | | | | |
| 202839_s_at | NADH dehydrogenase (ubiquinone) | NDUFB7 | −13.5 | 27.0 | 3.6E−03 | −25.0 | 3.3E−03 | 0 | 0 | 1 |
| | 1 beta subcomplex, 7, 18 kDa | | | | | | | | | |
| 201226_at | NADH dehydrogenase (ubiquinone) | NDUFB8 /// | −9.2 | 28.0 | 1.2E−03 | −16.4 | 1.4E−03 | 1 | 0 | 0 |
| | 1 beta subcomplex, 8, 19 kDa /// | SEC31B | | | | | | | | |
| | SEC31 homolog B | | | | | | | | | |
| 201966_at | NADH dehydrogenase (ubiquinone) | NDUFS2 | −15.0 | 22.8 | 6.4E−03 | −33.0 | 3.1E−03 | 0 | 0 | 1 |
| | Fe—S protein 2, 49 kDa (NADH- | | | | | | | | | |
| | coenzyme Q reductase) | | | | | | | | | |
| 218809_at | pantothenate kinase 2 | PANK2 | −16.6 | 25.1 | 7.5E−03 | −33.0 | 1.6E−03 | 1 | 0 | 0 |
| | (Hallervorden-Spatz syndrome) | | | | | | | | | |
| 200006_at | Parkinson disease (autosomal | DJ-1 | −12.9 | 24.9 | 3.0E−03 | −25.9 | 5.5E−04 | 0 | 0 | 1 |
| | recessive, early onset) 7 | | | | | | | | | |
| 209019_s_at | PTEN induced putative kinase 1 | PINK-1 | −18.6 | 31.4 | 1.6E−03 | −29.5 | 7.2E−03 | 1 | 0 | 0 |
| 209018_s_at | PTEN induced putative kinase 1 | PINK-1 | −17.5 | 24.2 | 6.9E−03 | −36.2 | 2.9E−03 | 1 | 0 | 0 |
| 224913_s_at | translocase of inner mitochondrial | TIMM50 | −21.0 | 27.8 | 7.2E−03 | −37.7 | 5.2E−03 | 1 | 0 | 0 |
| | membrane 50 homolog | | | | | | | | | |
| 218357_s_at | translocase of inner mitochondrial | TIMM8B | −9.6 | 23.6 | 2.6E−03 | −20.4 | 3.2E−03 | 0 | 0 | 1 |
| | membrane 8 homolog B | | | | | | | | | |
| 218190_s_at | ubiquinol-cytochrome c reductase | UCRC | −9.4 | 21.2 | 8.4E−03 | −22.1 | 1.2E−04 | 1 | 0 | 0 |
| | complex (7.2 kD) | | | | | | | | | |
| 208909_at | ubiquinol-cytochrome c reductase, | UQCRFS1 | −14.0 | 24.5 | 9.1E−03 | −28.6 | 1.4E−03 | 1 | 0 | 0 |
| | Rieske iron-sulfur polypeptide 1 | | | | | | | | | |

CC-CT (%) are the differences in average expression in age-matched groups.
CC-CT (years) were calculated by averaging molecular-chronological year deviations for a gene in age-matched groups (see Section VI-E).
Age expression differences and p-values were determined from age regression lines (see Section I-E).
N (neuronally-enriched expression),
G (Glial-enriched expression),
B (expressed to similar levels in both neurons and glia) (see Section III-C for methods of determining cellular origin of transcript changes.

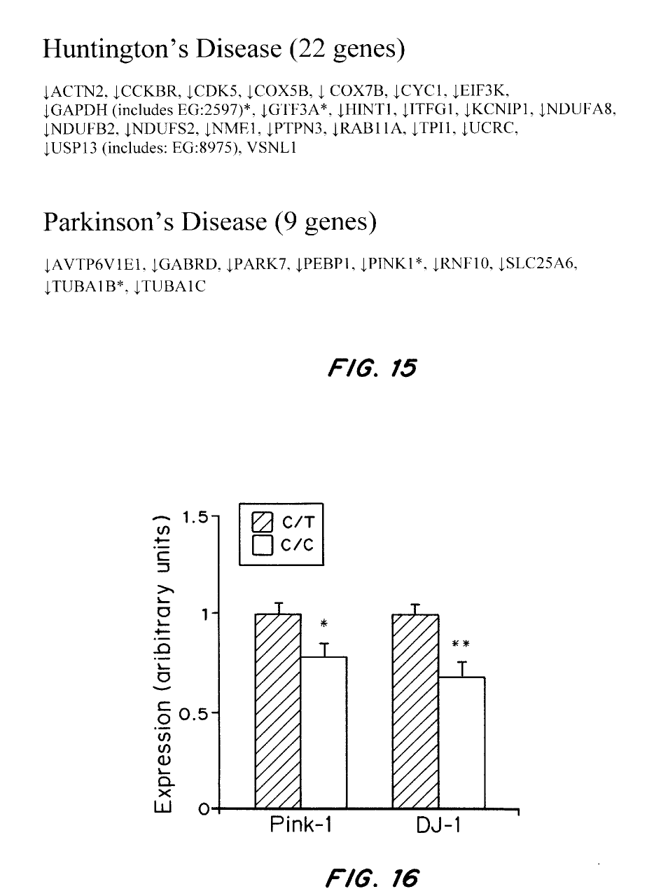

Strikingly, considering SIRT5's mitochondrial localization (Gan, et al., Neuron, 58:10-4 (2008); Nakagawa, et al., Cell, 137:560-70 (2009)), was that many core transcripts coded for mitochondrial-localized proteins, including numerous components of the electron transport chain (FIG. 11C). The top two identified canonical pathways were mitochondrial dysfunction and oxidative phosphorylation, and the top functional categories—genetic and neurological diseases—were predominated by two diseases linked to mitochondrial dysfunction: Parkinson's (9 associated genes) and Huntington's (22 associated genes) (FIG. 11C, FIGS. 10-15). Most directly, SIRT5ₚᵣₒₘ₂ genotype accounted for all age-related declines in expression of the familial Parkinson's genes, PINK1 and DJUPARK7 (FIG. 11D; qPCR-validated, FIG. 16). Representative core transcript age-regressions (PD genes) by SIRT5ₚᵣₒₘ₂ genotype are shown in FIG. 11D, and multi-hit model of age onset is shown in FIG. 11E. Rates of age-regulated changes in disease gene expression are accelerated in subjects carrying “risk alleles” of age-modulatory gene polymorphisms (i.e., SIRT5) in a brain area specific manner, resulting in this case in earlier age at which decreased expression reaches a critical theoretical threshold for symptom or disease onset. Conversely, protective factors (genetic and/or environmental) would delay onset. A similar mechanism would occur for age upregulated disease-related genes. People with loss of expression/function mutations in these genes develop early onset Parkinson's (Schapira, Lancet Neurol., 7:97-109 (2008)). Together, these findings suggest that SIRT5ₚᵣₒₘ₂ may represent a novel indirect risk factor for mitochondria-related diseases, potentially including Parkinson's and Huntington's diseases.

### Example 5

### SIRT 5 SNP Distribution by Race

The Health, Aging and Body Composition (Health ABC) database (Atkinson, H., et al., J Gerontol A Biol Sci Med Sci, 62(8):844-850 (2007)) was chosen due to its large scale prospective investigation of multiple factors in subjects 65 years of age and older, consistent domain monitoring across studies and extensive expertise in the analysis of those data. After the Health ABC data base was stratified by race, the following results were obtained in the association in the SNP distribution by race (χ² 125.3323, P<0.0001): from the 2768 total population, 1642 (59.32%) were white and 1126 (40.68%) were black. Regarding the genotype distribution by race, among the white people, 772 (47.02%) were homozygote for the common allele, 699 (42.57%) were heterozygote and 171 (10.41%) were homozygotes with uncommon allele; among the black people, 764 (67.85%) were homozygotes for the common allele, 317 (28.15%) were heterozygotes and 45 (4%) were uncommon allele homozygotes (Table 15).

TABLE 15
Distribution of the SIRT 5 SNP in white and black population.

| SNP/ | | | | |
|---|---|---|---|---|
| RACE | C/C | C/T | T/T | TOTAL |
| WHITE | 47.02% | 42.57% | 10.41% | 100% |
| BLACK | 67.85% | 28.15% | 4% | 100% |

### Example 6

### Association with SIRT5 SNP with Models of Age of Onset

Based on the associations and correlations among the three functional outcomes of brain health (DSST, Gait Test and CES-D) and the independent variables (age and sex), the following models were run (Table 16). The results of each of the designed models are summarized in Table 17; the first five models were statistically significant: Model 1 (F=33.36, p<0.001), Model 2 (F=32.89, p<0.001), Model 3 (F=50.32, p<0.001), Model 4 (F=41.25, p<0.001), Model 5 (F=12.61, p<0.001). Model 6 was not statistically significant (F=0.64, p<0.526).

From the overall models (Table 17) only models 2 and 5 were significant for the SIRT5 SNP: in Model 2, people lost 0.94 units for every year of age and the emphasis on sex in the model made people increase 5.65 units, both changes in the DSST; in Model 5, the SIRT 5 SNP effect of sex in the model made people lose 0.09 units in the CES-D. The results from the rest of the Models (not statistically significant) were as follows: in model 1, people lost 0.72 units for every year of age and the emphasis on sex in the model made people increase 3.83 units in the DSST; in Model 3, people lost 0.01 units for every year of age and the emphasis of sex in the model made people lose 0.12 units in Gait test; and in model 4, people lost by 0.01 units for every year of age and the emphasis of sex in the model made people lose 0.13 units.

TABLE 16
Models designed for the association with SIRT5 SNP.

| No. | Outcome | Model | F-test | Significant |
|---|---|---|---|---|
| | DSST | | | |
| 1 | WHITE | DSST = β₀ + β₁ × GENOTYPE + β₂ × | 33.36, p < 0.001 | Yes |
| | | AGE + β₃ × SEX | | |
| 2 | BLACK | DSST = β₀ + β₁ × GENOTYPE + β₂ × | 32.89, p < 0.001 | Yes |
| | | AGE + β₃ × SEX | | |
| | Gait Test | | | |
| 3 | WHITE | Gait T = β₀ + β₁ × GENOTYPE + β₂ × | 50.32, p < 0.001 | Yes |
| | | AGE + β₃ × SEX | | |
| 4 | BLACK | Gait T = β₀ + β₁ × GENOTYPE + β₂ × | 41.25, p < 0.001 | Yes |
| | | AGE + β₃ × SEX | | |
| | CES-D | | | |
| 5 | WHITE | CES_D = β₀ + β₁ × GENOTYPE + β₂ × | 12.61, p < 0.001 | Yes |
| | | SEX | | |
| 6 | BLACK | CES_D = β₀ + β₁ × GENOTYPE | 0.64, p < 0.526 | No |
| | (ANOVA) | | | |

TABLE 17
Summary of results from the association with SIRT5 SNP.

| Model | | Model's | Parameter | | |
|---|---|---|---|---|---|
| Number | Outcome/Race | Variables | Estimate | t Value | Pr > \|t\| |
| 1 | DSST/White | Intercept | 88.6300 | 11.69 | <.0001 |
| | | RS938222 | 0.2495 | 0.58 | 0.5651 |
| | | AGE | −0.7255 | −7.15 | <.0001 |
| | | SEX | 3.8386 | 6.65 | <.0001 |
| 2 | DSST/Black | Intercept | 87.35 | 8.24 | <.0001 |
| | | RS938222 | 1.45 | 1.97 | 0.0486 |
| | | AGE | −0.9487 | −6.66 | <.0001 |
| | | SEX | 5.6574 | 6.82 | <.0001 |
| 3 | Gait Test/White | Intercept | 2.6148 | 16.77 | <.0001 |
| | | RS938222 | −0.0011 | −0.12 | 0.9017 |
| | | AGE | −0.0139 | −6.67 | <.0001 |
| | | SEX | −0.1250 | −10.55 | <.0001 |
| 4 | Gait Test/Black | Intercept | 2.6118 | 14.28 | <.0001 |
| | | RS938222 | 0.0065 | 0.52 | 0.6024 |
| | | AGE | −0.0161 | −6.56 | <.0001 |
| | | SEX | −0.1307 | −9.16 | <.0001 |
| 5 | CES-D/White | Intercept | 0.45136 | 1.62 | 0.1052 |
| | | RS938222 | −0.03185 | −2.04 | 0.0419 |
| | | AGE | 0.00035 | 0.10 | 0.9239 |
| | | SEX | 0.09711 | 4.63 | <.0001 |
| 6 | CES-D/Black | Intercept | 0.3333 | 1.02 | 0.3064 |
| | | RS938222 | −0.0165 | −0.72 | 0.4702 |
| | | AGE | 0.0039 | 0.90 | 0.3668 |
| | | SEX | 0.0099 | 0.39 | 0.6977 |

TABLE 18
Models 2 and 5 divided by their three different genotype components
(C/C, C/T, T/T).

| Model | | |
|---|---|---|
| No. | OUTCOME | MODEL |
| | DSST | |
| 2 | BLACK | DSST = β₀ + β₁ × GENOTYPE0 + β₂ × AGE + |
| | Population | β₃ × SEX |
| | | DSST = β₀ + β₁ × GENOTYPE1 + β₂ × AGE + |
| | | β₃ × SEX |
| | | DSST = β₀ + β₁ × GENOTYPE2 + β₂ × AGE + |
| | | β₃ × SEX |
| | CES-D | |
| 5 | WHITE | CES_D = β₀ + β₁ × GENOTYPE0 + |
| | Population | β₂ × SEX |
| | | CES_D = β₀ + β₁ × GENOTYPE1 + |
| | | β₂ × SEX |
| | | CES_D = β₀ + β₁ × GENOTYPE2 + |
| | | β₂ × SEX |

Results

From the Models 2 and 5 which were selected to be tested for their association among each of the three possible genotypes of SIRT5 SNP (C/C, C/T & TT), only Model 2 had found a borderline significant association in the genotype C/C vs. C/T (common homozygote vs. heterozygote) with a statistically significant difference of p=0.051 (Table 19); in other words, in the black population, the recessive allele “T” causes the DSST score to increase, or vice-versa. The dominant allele “C” makes the DSST score decrease. Model 5 had an association between the genotype C/C vs. C/T (common homozygotes vs. heterozygotes); however, the statistical significance was p=0.08 (Table 20).

TABLE 19
Comparison (T-test) among common homozygote C/C vs. heterozygote
C/T from Model 2. DSST in black population = β₀ + β₁ × GENOTYPE
(C/C, C/T) + β₂ × AGE + β₃ × SEX.

| Contrast | SE | t-value | P-value |
|---|---|---|---|
| Most Frequent | 0.956 | 1.950 | 0.051 |
| Homozygote vs. | | | |
| Heterozygote | | | |

TABLE 20
Comparison (T-test) among common homozygote C/C vs. heterozygote
C/T from Model 5. CES_D = β₀ + β₁ × GENOTYPE
(C/C, C/T) + β₂ × SEX.

| Contrast | SE | t-value | P-value |
|---|---|---|---|
| Most Frequent | 0.0352 | 1.73 | 0.0830 |
| Homozygote vs. | | | |
| Heterozygote | | | |

The C/C SIRT5 genotype was associated here with (1) lower DSST scores in the black population (almost 2 units lower than heterozygotes) and (2) displayed trend-level higher CES-D “depressive-like” scores in the white population, hence supporting the SIRT5 C/C genotype as a risk factor for both biological brain age and related functional outcomes.

The analysis of covariates showed that females tend to have higher DSST scores than males; while in Gait scores the tendency is the opposite. Also according to our expectations, this analysis shows that older populations have the propensity for lower scores in DSST and higher Gait scores. As for CES-D scores, the results showed that white females tend to report more depressive symptoms than males. In DSST heterozygotes, members of the black population are more likely to have almost 2 units more than common homozygotes.

The more SIRT5 “C” alleles people carry, the greater the risk for a lower DSST score. Alternatively, people who carry the allele “T” have a “protective” factor to avoid poor DSST scores; for example, heterozygous people having C/T showed higher DSST scores than the most common homozygote individuals regarding their genotype SIRT5 SNP.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method for determining a subject's risk or propensity of developing a neurological disease or disorder, comprising:
determining the SIRT5ₚᵣₒₘ₂ (rs9382222) genotype in a sample obtained from the subject, wherein the presence of the SIRT5ₚᵣₒₘ₂ (rs9382222) C/C genotype is indicative of an increased risk of developing a neurological disease or disorder relative to a subject with an SIRT5ₚᵣₒₘ₂ (rs9382222) C/T genotype.

2. The method of claim 1 wherein the neurological disease or disorder is related to mitochondrial dysfunction.

3. The method of claim 1 wherein the neurological disease or disorder is Huntington's disease.

4. The method of claim 1 wherein the neurological disease or disorder is Parkinson's disease.

5. The method of claim 1 wherein the neurological disease or disorder is schizophrenia.

6. The method of claim 1, wherein the SIRT5ₚᵣₒₘ₂ (rs9382222) genotype is detected by gene sequencing.

7. The method of claim 1, wherein the SIRT5ₚᵣₒₘ₂ (rs9382222) genotype is detected by allele specific hybridization.

8. The method of claim 1, wherein a treatment protocol is selected for the subject based on the presence of the SIRT5ₚᵣₒₘ₂ (rs9382222) C/C genotype.

9. A method for determining a subject's risk or propensity of developing a neurological disease or disorder, comprising:
determining the SIRT5ₚᵣₒₘ₂ (rs9382222) genotype in a sample obtained from the subject, wherein the presence of SIRT5ₚᵣₒₘ₂ (rs9382222) C/T genotype is indicative of a reduced risk of developing a neurological disease or disorder relative to a subject with an SIRT5ₚᵣₒₘ₂ (rs9382222) C/C genotype.

10. The method of claim 9 wherein the neurological disease or disorder is Huntington's disease.

11. The method of claim 9 wherein the neurological disease or disorder is Parkinson's disease.

12. The method of claim 9 wherein the neurological disease or disorder is Alzheimer's disease.

13. The method of claim 9 wherein the neurological disease or disorder is selected from the group consisting of schizophrenia, bipolar disorder, and amyotrophic lateral schlerosis.

14. A nucleic acid probe comprising at least 14 nucleotides that specifically hybridizes under stringent conditions to an SIRT5ₚᵣₒₘ₂ (rs9382222) C allele or T allele.

15. The nucleic acid probe of claim 14, wherein the nucleic acid probe is a molecular beacon.

16. The nucleic acid probe of claim 14, wherein the nucleic acid probe is attached to a solid support.

17. The nucleic acid probe of claim 14, wherein the nucleic acid probe is a component of a kit.

18. The nucleic acid probe of claim 14, wherein the nucleic acid probe is labeled with a detectable label.

19. The nucleic acid probe of claim 14 wherein the nucleic acid probe consists of 14-20 consecutive nucleotides of SEQ ID NO:1 spanning the cystidine at position 27 of SEQ ID NO:1, or a complement thereof, wherein the nucleic acid probe specifically hybridizes to SEQ ID NO:1 under stringent conditions.

20. The nucleic acid probe of claim 14 wherein the nucleic acid probe consists of 14-20 consecutive nucleotides of SEQ ID NO:2 spanning the thymidine at position 27 of SEQ ID NO:2, or a complement thereof, wherein the nucleic acid probe specifically hybridizes to SEQ ID NO:2 under stringent conditions.


SEQUENCE LISTING (2 sequences)

SEQ ID NO: 1 - DNA, 52 bp, Homo sapiens
ccactaaactccctcctacccccacccaataactatggacaactttccatcc

SEQ ID NO: 2 - DNA, 52 bp, Homo sapiens
ccactaaactccctcctacccccacctaataactatggacaactttccatcc